(19) United States
(12) Patent Application Publication
(10) Pub. No.: US 2008/0171773 A1
(43) Pub. Date: Jul. 17, 2008

(54) FACTOR Xa INHIBITOR

(75) Inventors: Jeremy John Edmunds, Acton, MA (US); Brian Matthew Samas, New London, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/947,713

(22) Filed: Nov. 29, 2007

Related U.S. Application Data
(60) Provisional application No. 60/867,714, filed on Nov. 29, 2006.

(51) Int. Cl. A61K 31/4439; C07D 401/02; A61P 7/00; A61P 35/04

(52) U.S. Cl. 514/341; 546/275.4

(57) ABSTRACT

(R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide} pharmaceutical compositions, methods and methods of using the compound or pharmaceutical compositions thereof to treat diseases characterized by abnormal thrombosis in mammals. Crystalline form of (R)-5-Methyl-4,5-dihydro-pyrazole-1,5-dicarboxylic acid 1-[(4-chloro-phenyl)amide]5-{[2-fluoro-4-(2-oxo-2H-pyridin-1-yl)-phenyl]-amide}

This application claims the benefit of Provisional Application No. 60/867,714, filed Nov. 29, 2006.

### TECHNICAL FIELD

The present invention relates to a Factor Xa inhibitor enantiomer, pharmaceutical compositions comprising the enantiomer and methods of using them as therapeutic agents for treating diseases, characterized by abnormal thrombosis, in mammals.

### BACKGROUND OF THE INVENTION

Ischemic heart disease and cerebrovascular disease are the leading causes of death in the world. Abnormal coagulation and inappropriate thrombus formation within blood vessels precipitate many acute cardiovascular diseases.

Thrombin can be considered the key or principal regulatory enzyme in the coagulation cascade; it serves a pluralistic role as both a positive and negative feedback regulator in normal hemostasis. However, in some pathologic conditions, the positive feedback regulation is amplified through catalytic activation of cofactors required for thrombin generation. Such cofactors include factor Xa, a serine protease that occupies a pivotal position in the coagulation cascade.

Abnormal coagulation and inappropriate thrombus formation within blood vessels precipitates many cardiovascular diseases such as myocardial infarction, myocardial ischemia, stroke in association with atrial fibrillation, deep venous thrombosis (DVT), pulmonary embolism, cerebral ischemia or infarction, peripheral artery disease, restenosis, atherosclerosis and thromboembolism. In addition, thrombosis has been linked with non-cardiovascular diseases such as cancer, diabetes, chronic obstructive pulmonary disease (COPD) and sepsis. Currently some of these conditions are treated with anti-thrombotic agents. However, many of these agents require close monitoring of the patient to protect against bleeding. Recently, it has been appreciated that factor Xa inhibition may provide sustained antithrombotic protection. In animal studies, short term exposure to factor Xa inhibitors produces a sustained antithrombotic effect. Data indicate that factor Xa inhibition potentially provides a large therapeutic window between antithrombotic efficacy and bleeding tendency. Consequently, there may exist a range in which factor Xa inhibition is achieved without a concurrent increase in a patients' susceptibility to bleeding, unlike currently available drugs.

Sepsis is a complex extension of acute inflammation and involves a cycle of progressive amplification of coagulation and inflammation. The intimate involvement of the coagulation system in the progression of this disease has led to treatments that include antithrombotic agents. However, currently available antithrombotic agents do no provide adequate treatment of the disease.

There is a well-known connection between malignancy and thrombosis. Recent evidence has shown that Factor Xa plays a role in tumor metastasis independent from its role in thrombosis and hemostasis.

Recent evidence has indicated that factor Xa, can influence many cellular responses acting via proteolytically activated receptors (PARs). PARs have been shown to have an indirect role in chronic obstructive pulmonary disease COPD.

Type 2 diabetic patients without previous clinical coronary artery disease have the same probability of dying from coronary disease as non-diabetic subjects who have had a previous myocardial infarction. The increased cardiovascular risk in diabetes is contributed to by the clustering of cardiovascular risk factors, which include hypertension, dyslipidemia, hyperinsulinemia, hyperglycemia, obesity, and haemostatic risk factors such as hyperfibrinogenemia and increased levels of plasminogen activator inhibitor-1. These risk factors combine to yield life-threatening thrombotic conditions that could effectively be reduced by treatment with Factor Xa inhibitors.

Therefore it is readily apparent that there still exists a need for more effective agents that regulate factor Xa proteolytic activity.

### SUMMARY OF THE INVENTION

The present invention encompasses (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide} and pharmaceutical compositions thereof. The invention also relates to methods of using the compound of the invention to treat diseases characterized by abnormal thrombosis in mammals. The invention further relates to a crystalline form of (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide.

The formula of (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide} is shown below.

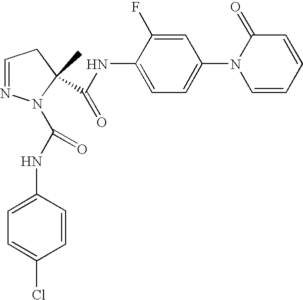

One embodiment of the invention is the compound (R)-5-Methyl-4,5-dihydro-pyrazole-1,5-dicarboxylic acid 1-[(4-chloro-phenyl)amide] 5-{[2-fluoro-4-(2-oxo-2H-pyridin-1-yl)-phenyl]-amide} wherein the (R) enantiomer is substantially pure.

Another embodiment of the invention is pharmaceutical composition comprising a pharmaceutically acceptable carrier, excipient or diluent and (R)-5-Methyl-4,5-dihydro-pyrazole-1,5-dicarboxylic acid 1-[(4-chloro-phenyl)-amide] 5-{[2-fluoro-4-2H-oxo-2H-pyridin-1-yl)-phenyl]-amide} wherein the (R) enantiomer is substantially pure.

Another embodiment of the invention is a method for the treatment of a thrombotic, or embolic disorder in a patient in need thereof, comprising administering a therapeutically effective amount of (R)-5-Methyl-4,5-dihydro-pyrazole-1,5-dicarboxylic acid 1-[(4-chloro-phenyl)-amide] 5-{[2-fluoro (2-oxo-2H-pyridin-1-yl)-phenyl]-amide} wherein the (R) enantiomer is substantially pure.

Another embodiment of the invention is a method for the treatment of a patient with a metastatic disorder in order to prolong survival in a patient in need thereof comprising administering a therapeutically effective amount of (R)5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]amide} wherein the (R) enantiomer is substantially pure.

Another embodiment of the invention is a method for the treatment of sepsis in a patient in need thereof, comprising administering a therapeutically effective amount of (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl] amide} wherein the (R) enantiomer is substantially pure.

Another embodiment of the present invention is the crystalline Form A of (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide} (Form A). Form A is characterized by the calculated x-ray powder diffraction (calculated PXRD) pattern (FIG. 4).

### BRIEF DESCRIPTION OF THE DRAWING

FIG. 1. Elution pattern from chiral chromatography of the racemate as a function of time.

FIG. 2. Elution pattern, after chiral separation, of the (R) enantiomer as a function of time.

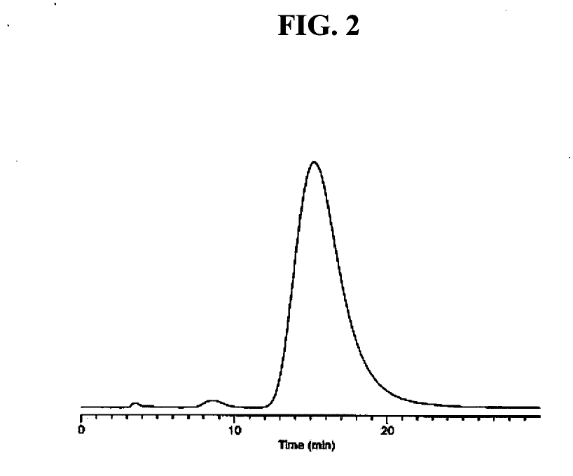

FIG. 3. Elution pattern from chiral chromatography of the chirally synthesized (R) enantiomer as a function of time.

FIG. 4. Diffractogram of the calculated PXRD of Form A

FIG. 5. Diffractogram of the Experimental PXRD of Form A

FIG. 6. Overlaid Diffractograms of calculated PXRD and Experimental PXRD or Form A

### DETAILED DESCRIPTION OF THE INVENTION

### Definitions

Patient includes humans as well as other mammals.

Treatment includes therapeutic and/or prophylactic treatment.

Prophylactic treatment means treatment to prevent or lessen the risk of a thrombotic disorder in a patient in need thereof.

Therapeutic treatment means treatment of an existing disorder in a patient in need thereof.

A patient may require therapeutic and prophylactic treatment. One of skill in the art would know when treatment should be administered and how a patient with a thrombotic disorder should be treated with the compound of the present invention.

“DVT” means deep vein thrombosis. PE means pulmonary embolism. VTE means venous thromboembolism.

“Primary DVT” means DVT occurring in a patent with no prior history of DVT.

“Secondary VTE” means recurrence of DVT or PE in a patient with prior history of DVT or PE, or occurrence of PE in a patient with a prior history of DVT.

“Substantially pure” refers to a preparation of 5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide} where in the (R) enantiomer is present in excess of the (S) enantiomer.

The terms “(R)-5-Methyl-4,5-dihydro-pyrazole-1,5-dicarboxylic acid 1-[(4-chloro-phenyl)amide] 5-{[2-fluoro-4-(2-oxo-2H-pyridin-1-yl)-phenyl]-amide}” and “(R) enantiomer” are used interchangeably.

The terms “(S)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide}” and “(S) enantiomer” are used interchangeably.

An arterial embolism includes but is not limited to pulmonary embolism, myocardial infarction, and cerebral infarction.

A venous embolism includes but is not limited to deep vein thrombosis and intra-abdominal vein thrombosis.

The term “excipient” is used herein to describe any ingredient other than the compound of the invention. The choice of excipient will to a large extent depend on the particular mode of administration. The term “polymorph” and “crystalline polymorph” and “crystalline form” are used interchangeably herein.

The term “polymorphic form” and “polymorph” are used interchangeably herein.

“Form A”, “Form A polymorph”, “crystalline form A” and “Form A polymorph of (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide} mean the same and are used interchangeably herein.

The term “crystalline form,” “polymorphic form” or “polymorph” as applied to (1,2-Pyrrolidinedicarboxamide, (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide} refers to a solid state form wherein the molecules of 1(R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide}, are arranged to form a distinguishable crystal lattice yielding characteristic diffraction peaks when subjected to X-ray radiation.

When used in conjunction with PXRD, the term “pattern” and “diffractogram” as used herein, have the same meaning.

“Calculated PXRD” and “predicted PXRD” mean the same and are used interchangeably herein.

Experimental PXRD and Powder PXRD mean the same and are used interchangeably herein.

A compound may exist in different physical forms. For example, these compounds may exist as solids, liquids, or gases. Solid forms may be amorphous or may exist as distinct crystalline forms. Different crystalline forms often have different physical properties (i.e. bioavailability, solubility, melting points, etc).

These different crystalline forms are called polymorphs. One method of determining the structure of a polymorph is single crystal X-ray analysis. In this analysis the crystalline state of a compound is described by several crystallographic parameters including unit cell dimensions, space group, and atomic position of all atoms in the compound relative to the origin of its unit cell. A more detailed discussion of single crystal X-ray analysis may be found in International Tables for X-ray Crystallography, Vol. IV, pp. 55, 99, 149 Birmingham: Kynoch Press, 1974, G. M. Sheldrick, SHELXTL. User Manual, Nicolet Instrument Co., 1981 and in Crystal Structure Analysis by Glusker, and Trueblood, 2nd ed.; Oxford University press: New York, 1985.

Single crystal X-ray analysis is where one crystal is placed in the X-ray beam. Data generated in determining the structure of a single crystal allows one skilled in the art to calculate a powder X-ray diffraction pattern (calculated PXRD). This conversion is possible because the single crystal experiment routinely determines the unit cell dimensions, space group, and atomic positions. These parameters provide a basis to calculate the powder pattern for a particular polymorph.

Another method for determining the structure of a polymorph is powder X-ray diffraction (PXRD). PXRD analysis involves collection of crystallographic data from a group of crystals. To perform PXRD analysis, a powdered sample of the crystalline material is placed in a holder that is then placed into a diffractometer. An X-ray beam is directed at the sample, initially at a small angle relative to the plane of the holder, and then moved through an arc that continuously increases the angle between the incident beam and the plane of the holder. The intensity of the reflected radiation is recorded. These data can be expressed in graphical form as a PXRD pattern.

Measurement differences associated with such X-ray powder analyses result from a variety of factors including: (a) errors in sample preparation (e.g. sample height), (b) instrument errors (e.g. flat sample errors), (c) calibration errors, (d) operator errors (including those errors present when determining the peak locations), (e) the nature of the material (e.g. preferred orientation and transparency errors), (f) compound lot to lot differences and (g) machine type. Calibration errors, sample height errors, lot-to-lot variations, and machine type differences often result in a shift of all the peaks in the same direction. These shifts can be identified from the X-ray diffractogram and can be eliminated by compensating for the shift (applying a systematic correction factor to all peak position values) or recalibrating the instrument. This correction factor is, in general, in the range of 0 to 0.2 degrees 2θ.

One embodiment of the invention is the compound (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide}, wherein the (R) enantiomer is substantially pure, preferably wherein the ratio of the (R) enantiomer to the (S) enantiomer is greater than 4:1, more preferably 17:3, more preferably 9:1, more preferably 19:1, most preferably 99:1.

One embodiment of the invention is pharmaceutical composition comprising a pharmaceutically acceptable carrier, excipient or diluent and (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide} wherein the (R) enantiomer is substantially pure, preferably wherein the ratio of the (R) enantiomer to the (S) enantiomer is greater than 4:1, more preferably 17:3, more preferably 9:1, more preferably 19:1, most preferably 99:1.

Another embodiment of the invention is a method for the treatment, which includes prophylactic treatment and therapeutic treatment, of a thrombotic, or embolic disorder in a patient in need thereof, comprising administering a therapeutically effective amount of (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)phenyl]-amide} wherein the (R) enantiomer is substantially pure, preferably wherein the ratio of the (R) enantiomer to the (S) enantiomer is greater than 4:1, more preferably greater than 17:3, more preferably 9:1, more preferably 19:1 and most preferably 99:1. Thrombotic or embolic disorder includes, but is not limited to, primary or secondary venous thrombosis, arterial thrombosis, venous embolism, arterial embolism, pulmonary embolism, pulmonary hypertension, venous stenosis, venous restenosis, arterial stenosis, arterial restenosis, atrial fibrillation, stroke, angina, diabetes, cancer, heart failure, or immobilization due to trauma, surgery or medical illness.

Another embodiment of the invention is method for the treatment of a patient with a metastatic disorder in order to prolong survival in a patient in need thereof comprising administering a therapeutically effective amount of comprising (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide} wherein the (R) enantiomer is substantially pure preferably wherein the ratio of the (R) enantiomer to the (S) enantiomer is greater than 4:1, more preferably 17:3, more preferably 9:1, more preferably 19:1, most preferably 99:1.

Another embodiment of the invention is a method for the treatment of sepsis in a patient in need thereof, comprising administering a therapeutically effective amount of administering a therapeutically effective amount of comprising (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide} wherein the (R) enantiomer is substantially pure preferably wherein the ratio of the (R) enantiomer to the (S) enantiomer is greater than 4:1, more preferably 17:3, more preferably 9:1, more preferably 19:1, most preferably 99:1.

Another embodiment of the invention is the use of substantially pure (R-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide for the manufacture of a medicament for the therapeutic treatment or prophylactic treatment of thrombotic disorders in mammals, preferably wherein the ratio of the (R) enantiomer to the (S) enantiomer is greater than 4:1, more preferably 17:3, more preferably 9:1, more preferably 19:1, most preferably 99:1.

Another embodiment of the invention is a method for making (R)-5-Methyl-4,5-dihydro-pyrazole-1,5-dicarboxylic acid 1-[(4-chloro-phenyl)-amide] 5-{([2-fluoro-4(2-oxo-2H-pyridin-1-yl)-phenyl] comprising:

Step (a)

(1) reacting the compound of Formula 11 with methacryoyl chloride in the presence of a solvent and a base; or

(2) reacting a compound of Formula 11 with lithium chloride, triethylamine and 2-methylacrylic anhydride;

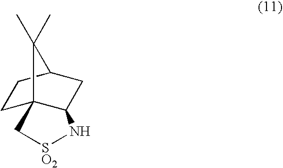

to give a compound of Formula 12

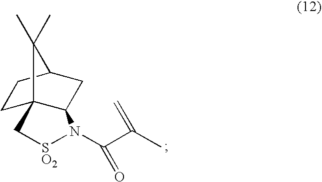

Step (b) reacting the compound of Formula 12 with trimethylsilyl diazomethane, treated with dilute acid and isolated to afford a compound of Formula 13

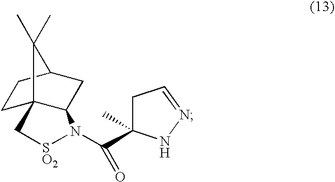

Step (c) treating the compound of Formula 13 with an isocyanate and a mild base in a solvent to give a compound of Formula 14

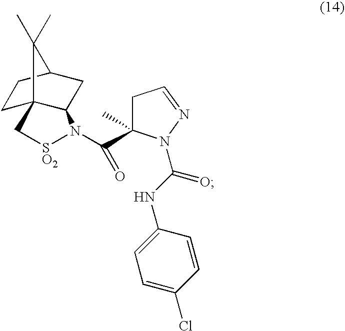

Step (d) removing the chiral auxiliary to give a compound of Formula 15

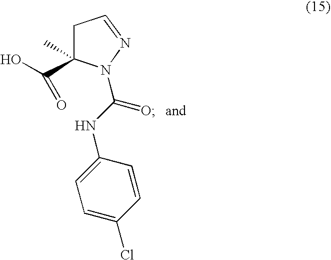

Step (e) coupling the compound of Formula 15 with a compound of Formula 3

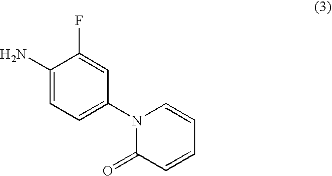

to afford the desired compound.

Another embodiment of the present invention is the crystalline Form A of (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide} Form A is characterized by the x-ray powder diffraction (PXRD) pattern (Table 4).

Other embodiments of the present invention include, but are not limited to: A crystalline form having a powder X-ray diffraction pattern with at least one peak at 5.4, 7.2, 9.4, 10.9, 15.6, 19.6. 21.7 or 23.3 degrees 2θ;

A crystalline form having a powder X-ray diffraction pattern with peaks at 19.6 and 21.7 and one or more additional peaks at 7.2, 9.4, 10.9, 15.6 or 23.3 degrees 2θ;

A crystalline form having a powder X-ray diffraction pattern with peaks at peaks at 10.9, 19.6 and 21.7 and one or more additional peaks at 7.2, 9.4, 15.6 or 23.3 degrees 2θ.

### Activity Determination

The ability of compounds to act as inhibitors of human factor Xa catalytic activity can be assessed by determination of the concentration of test substance that inhibits by 50% (IC₅₀) the ability of human factor Xa to cleave the chromogenic substrate S2765 (N-CBz-D-Arg-L-Gly-L-Arg-p-nitroanilide. 2HCl).

### Formulations

### Drug Substance

The compound of the invention may exist in a continuum of solid states ranging from fully amorphous to fully crystalline. The compound of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water. The compound of the invention may also exist in a mesomorphic state (mesophase or liquid crystal) when subjected to suitable conditions. For more information, see Crystals and the Polarizing Microscope by N. H. Hartshorne and A. Stuart, 4^{th} Edition (Edward Arnold, 1970).

Conventional techniques for the preparation/isolation of individual enantiomers include chiral synthesis from a suitable optically pure precursor or resolution of the racemate using, for example, chromatography with chiral phases including but not limited to, simulated Moving Bed (SMB), chiral high pressure liquid chromatography; formation of diasteromeric salts with suitable chiral acids or bases, and enatiomeric enrichment through crystallization.

### Formulations

Pharmaceutical compositions suitable for the delivery of compound of the present invention and methods for their preparation will be readily apparent to those skilled in the art. Such compositions and methods for their preparation may be found, for example, in Remington's Pharmaceutical Sciences, 19th Edition (Mack Publishing Company, 1995). The compound of the present invention may be administered by any suitable route. The compound and compositions, for example, may be administered orally, rectally, parenterally, or topically. The compound of the invention may be formulated to be immediate and/or modified release. Modified release formulations include delayed-, sustained-, pulsed-, controlled-, targeted and programmed release.

### Oral Administration

The compound of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include, for example, solid, semi-solid and liquid systems such as tablets; soft or hard capsules containing multi- or nano-particulates, liquids, or powders; lozenges (including liquid-filled); chews; gels; fast dispersing dosage forms; films; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compound of the invention may also be used in fast-dissolving, fast-disintegrating dosage forms such as those described in Expert Opinion in Therapeutic Patents, 11(6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients, for example, anti-oxidants, colourants, flavoring agents, preservatives and taste-masking agents may be included.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated. The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of the present invention, a film-forming polymer, a binder, a solvent, a humectant, a plasticiser, a stabiliser or emulsifier, a viscosity-modifying agent and a solvent. Some components of the formulation may perform more than one function. The film-forming polymer may be selected from natural polysaccharides, proteins, or synthetic hydrocolloids and is typically present in the range 0.01 to 99 weight %, more typically in the range 30 to 80 weight %. Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

### Parenteral Administration

The compound of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration, for example include, needle (including microneedle) injectors, needle-free injectors, infusion techniques and stents.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

Compound of the invention may be formulated as a suspension or as a solid, semi-solid, or thixotropic liquid for administration as an implanted depot providing modified release of the compound of the present invention. Examples of such formulations include drug-coated stents and semi-solids and suspensions comprising drug-loaded poly(dl-lactic-coglycolic)acid (PGLA) microspheres.

### Topical Administration

The compound of the invention may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88(10), 955-958, by Finnin and Morgan (October 1999).

### Inhaled/Intranasal Administration

The compound of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

### Dosage

The compound of the present invention can be administered to a patient at dosage levels in the range of 0.1 to 2,000 mg per day. In another embodiment the compound of the present invention is administered to a patient in the range of 0.01 to 300 mg per day. In another embodiment, the compound of the present invention is administered to a patient at dosage levels in the range of 0.01 to 150 mg per day. In another embodiment, the compound of the present invention is administered to a patient at dosage levels in the range of 0.1 to 100 mg per day. 0.1-50 mg per day 0.1-25 mg per day 0.01-10 mg per day. The specific dosage used can vary. For example, the dosage can depend on a numbers of factors including the requirements of the patient, the condition being treated. Determination of optimum dosages for a particular patient is well-known to those skilled in the art.

### Co-administration

The compound of the present invention can be used, alone or in combination with other therapeutic agents, in the treatment of various conditions or disease states. The compound of the present invention and other therapeutic agent(s) may be administered simultaneously (either in the same dosage form or in separate dosage forms) or sequentially. The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration. The phrases “concurrent administration,” “co-administration,” “simultaneous administration,” and “administered simultaneously” means that the compounds are administered in combination.

In one embodiment, the compound of the present invention may be co-administered with an oral antiplatelet agent, including, but not limited to, dipyridamole, cilostazol and anegrilide hydrochloride. In still another embodiment, the compound of the invention may be co-administered with aspirin.

In another embodiment, the compound of the present invention may be co-administered with a glycoprotein IIb/IIIa inhibitor, including, but not limited to, aboiximab, eptifibatide and tirofiban. In still another embodiment, the compound of the invention may be co-administered with eptifibatide. In another embodiment, the compound of the invention may be co-administered with an investigational compound useful in treating platelet aggregation including, but not limited to, BAY 59-7939, YM-60828, M-55532, M-55190, JTV-803 and DX-9065a.

While embodiments of the invention have been illustrated or described, it is not intended that these embodiments illustrate and describe all possible embodiments of the invention. Rather, the words used in the specification are words of description rather than limitation, and it is understood that various changes may be made without departing from the spirit and scope of the invention.

### General Synthetic Schemes

U.S. Patent Application No. US 2003/0162787 to Bigge et al., discloses general synthetic schemes for synthesis of 5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide}.

The starting materials used herein are commercially available or may be prepared by routine methods known in the art (such as those methods disclosed in standard reference books such as the COMPENDIUM OF ORGANIC SYNTHETIC METHODS, Vol. I-VI (published by Wiley-Interscience)). The compound of the present invention may be prepared using the methods illustrated in the general synthetic schemes and experimental procedures detailed below. The general synthetic schemes are presented for purposes of illustration and are not intended to be limiting.

One method for the preparation of (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide} is depicted in Scheme A.

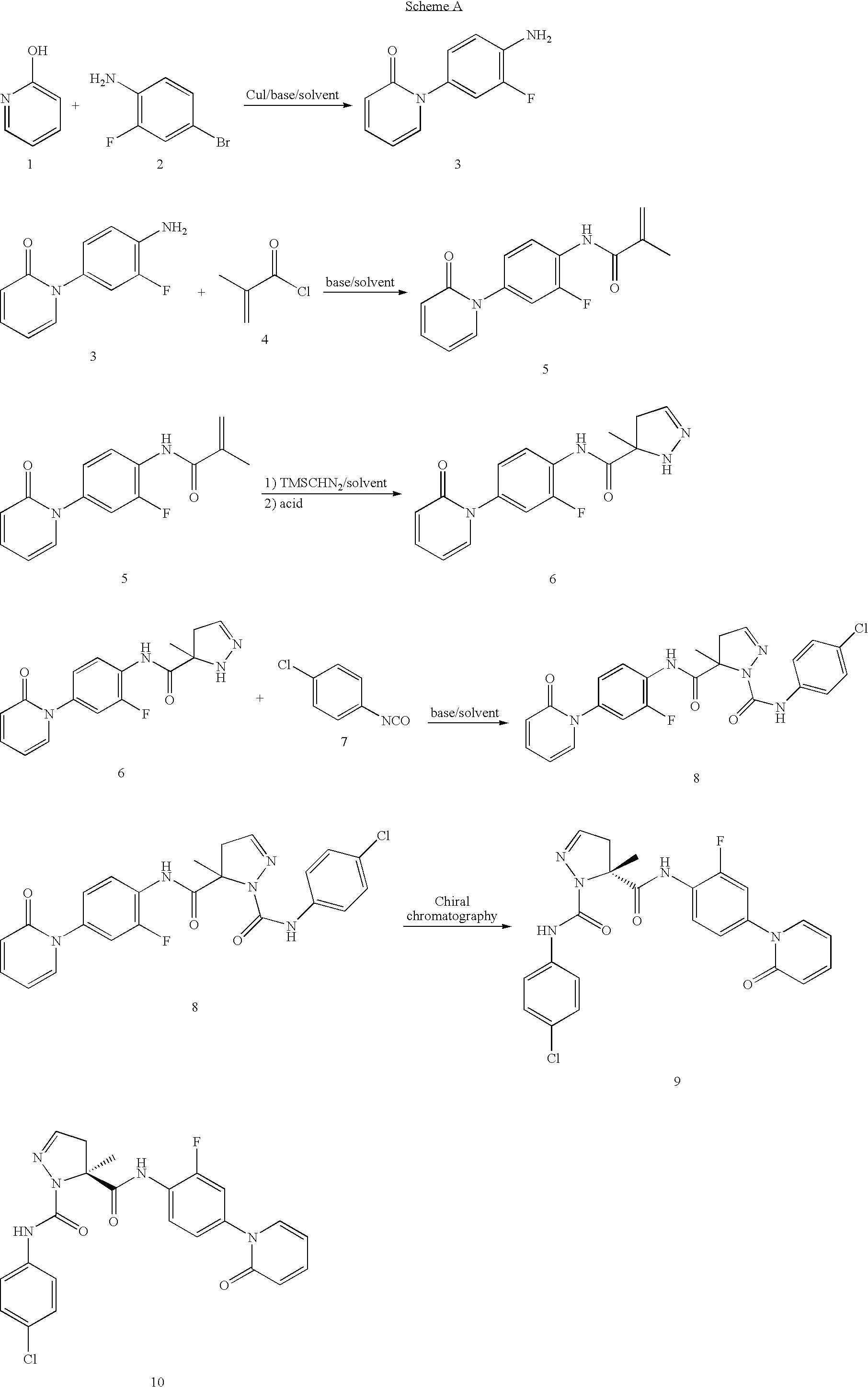

1-H-pyridin-2-one (1) is combined with 2-fluoro-4-bromoaniline and a base such as potassium carbonate, and Cul in a solvent such as DMF to yield 1-(4-amino-3-fluoro-phenyl)-1H-pyridine-2-one (3). The aniline (3) is converted to an acrylamide (5) by the addition of methacryoyl chloride (4) and a base, for example, saturated aqueous sodium bicarbonate or potassium carbonate, in a solvent such as tetrahydrofuran or ethylacetate at a temperature between about 0° C. to room temperature. The trimethylsilyl diazomethane is added to acrylamide (5) in a solvent such as chloroform, methylene chloride or ethylacetetate. After addition of acid, such as trifluoroacetic acid, acetic acid, or hydrofluoric acid the pyrazoline (6) is isolated. The pyrazoline is then allowed to react with an isocyanate (7) in the presence of an amine base such as triethylamine in a solvent such as chloroform to afford a racemic mixture (8) of the required compound. The mixture of enantiomers is then separated by chromatography, e.g. on a Chiralpak AS column®, in acetonitrile/methanol to afford the (R) enantiomer (9) and the (S) enantiomer (10).

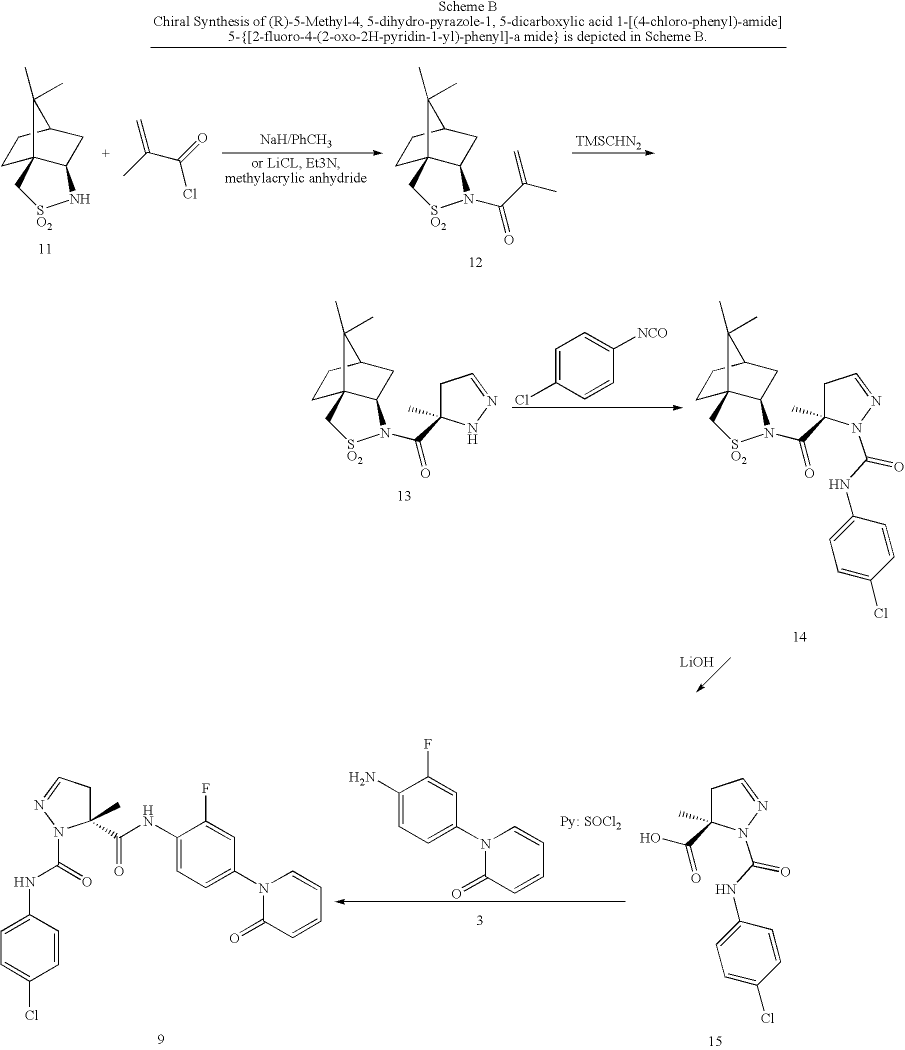

To a solution of the (1S)-(−)-2,10-camphorsultam (11) in a suitable solvent such as toluene was added sodium hydride and the mixture stirred. Methacryoyl chloride is added directly to the reaction mixture and then the product (12) is isolated by extraction with ethylacetate. Alternatively a solution of the sultam in THF is mixed with lithium chloride, triethylamine and 2-methylacrylic anhydride at −20° C. to room temperature and then filtered to afford the desired adduct (12) in a manner similar to that described by Ho, Guo-Jie; Mathre, David J. Lithium-Initiated Imide Formation. A Simple Method for N-Acylation of 2-Oxazolidinones and Bornane-2,10-Sultam. Journal of Organic Chemistry (1995), 60(7), 2271-3. This methacryloyl sultam adduct (12) is added to trimethylsilyl diazomethane and the mixture stirred for several days, in a manner similar to that described by Mish, Michael R.; Guerra, Francisco M.; Carreira, Erick M. Asymmetric Dipolar Cycloadditions of Me3SiCHN2. Synthesis of a Novel Class of Amino Acids: Azaprolines. Journal of the American Chemical Society (1997), 119(35), 8379-8380. Upon treatment with dilute acid, such as trifluoroacetic acid or hydrofluoric acid the pyrazoline (13) is isolated by chromatography. Upon treatment with an isocyanate, and mild base such as aqueous bicarbonate and stirring in a suitable solvent such as methylene chloride the urea adduct (14) is isolated. The chiral auxiliary is removed by hydrolysis with aqueous base such as lithium hydroxide to form (15). The carboxylic acid is then coupled with the aniline (3) under dehydrating conditions, such as pyridine with thionyl chloride, to afford the required amide (9).

### Factor Xa Inhibitory Activity

Following synthesis and separation of the enantiomers, each of the enantiomers was assayed for its IC₅₀ as described in Example 3 results are shown below in Table I

TABLE 1

| | | standard | standard error | number of |
|---|---|---|---|---|
| | mean IC₅₀ μM | deviation | of the mean | replicates |
| (R) enantiomer | 0.000982 | 0.000231 | 0.000045 | 26 |
| (S) enantiomer | 0.141333 | 0.017556 | 0.010136 | 3 |

### Single Crystal X-ray Analysis

Single crystals of the (R) enantiomer were grown by heat/cool from a solution of nitromethane, resulting in single crystals appropriate for X-ray structure determination. A representative crystal was surveyed and data were collected at ambient temperature using an APEX (Bruker-AXS) diffractometer. All crystallographic calculations were facilitated by the SHELXTL software package. The crystal structure was solved and refined in the monoclinic space group, P2₁. The stereocenter was determined from the flack parameter. The flack parameter was −0.0134 for this structure vs. 0.81 for the inverted structure. The refined structure fits well to the data with a final R-index 5.45% and no missing or misplaced electron density observed in the final difference Fourier. The asymmetric unit from the crystal structure confirms the molecular structure and chirality of the (R) enantiomer.

Tables 2-8 provide the data obtained from the single crystal X-ray analysis.
Table 2 summarizes general information about the crystal structure and refinement.
Table 3 provides Atomic coordinates (10⁴) and equivalent isotropic displacement parameters (Å²×10³) for (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide}. U(eq) is defined as one third of the trace of the orthogonalized U^{ij} tensor.
Table 4 provides the Bond lengths [Å] and angles [°].
Table 5 provides Anisotropic displacement parameters (Å²×10³). The anisotropic displacement factor exponent takes the form: −2π²[h²a*²U¹¹+ . . . +2 h k a*b*U¹²]
Table 6 provides the Hydrogen coordinates (×10⁴) and isotropic displacement parameters (Å²×10³).
Table 7 provides the Torsion angles [°].
Table 8 depicts the Hydrogen bonds [Å and °]

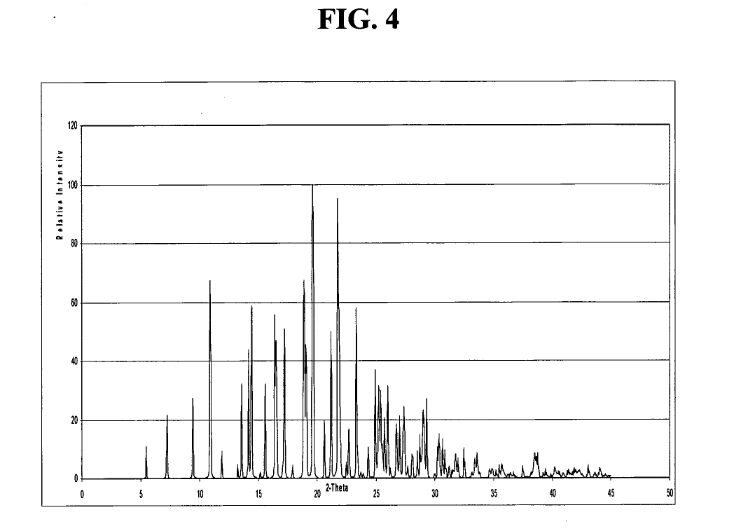

This data was used to generate a calculated powder pattern. The MS Modeling software suite version 4.0.0.0 by Accelrys Software (2004) generated the diffractogram show in figure FIG. 4. This calculated powder pattern was compared to the previous lots of bulk material as proof that this sample was representative of the polymorph.

### Experimental PXRD

Sample was tapped out of vial and pressed onto zero-background silicon in aluminum holder. Sample width 5 mm. Sample was stored and run at room temperature. Sample was spun at 40 rpm around vertical axis during data collection. The powder X-ray diffraction (PXRD) data were collected on a collected on a Rigaku (Tokyo, Japan) Ultima-plus diffractometer with CuKa radiation operating at 40 kV and 40 mA. A Nal scintillation detector detected diffraction radiation. Samples were scanned by continuous θ/2θ coupled scan from 3.00° to 45.00° in 2θ at a scan rate of 1°/min: 1.2 sec/0.02° step. Data were collected with an IBM-compatible interface equipped with 6 position autosampler, software=RigMeas v2.0 (Rigaku, December 1995) and Excel (Microsoft Office Excel 2003 Version 11.6355.6360) CuKₐ radiation (40 mA, 40 kV, λ=1.5419 Å). Slits I and II at 0.5°, slit III at 0.6°. Samples were collected at room temperature.

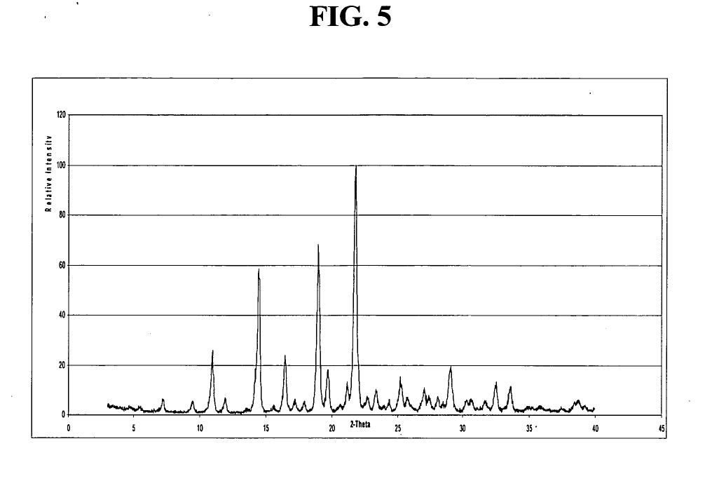

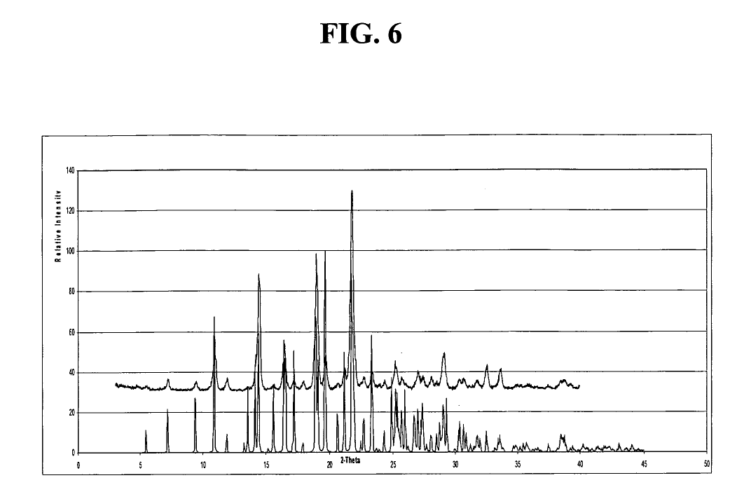

FIG. 5 shows the experimental PXRD diffractogram. An overlay of the experimental and calculated PXRD patterns is depicted in FIG. 6 where the bottom defractogram corresponds to the calculated PXRD and the top defractogram corresponds to the experimental PXRD.

Table 9 gives characteristic peaks identified in the calculated pattern.

TABLE 2

| | |
|---|---|
| Crystallization | Heat/cool of nitromethane solution |
| Empirical formula | C23H19ClFN5O3 |
| Formula weight | 467.88 |
| Temperature | 300(2) K |
| Wavelength | 0.71073 Å |
| Crystal system | Monoclinic |
| Space group | P2(1) |
| Unit cell dimensions | a = 16.2006(19) Å; b = 7.1436(8) Å; |
| | c = 18.773 (2) Å; α = 90°; |
| | β = 90.113(3)°; γ = 90° |
| Volume | 2172.6(4) Å³ |
| Z | 4 |
| Density (calculated) | 1.430 Mg/m³ |
| Absorption coefficient | 0.221 mm⁻¹ |
| F(000) | 968 |
| Crystal size | 0.50 × 0.05 × 0.03 mm³ |
| Theta range for data collection | 1.66 to 23.26° |
| Index ranges | −18 <= h <= 17, −7 <= k <= 5, |
| | −19 <= l <= 20 |
| Reflections collected | 9975 |
| Independent reflections | 4961 [R(int) = 0.0471] |
| Completeness to theta = 23.26° | 99.9% |
| Absorption correction | SADABS |
| Max. and min. transmission | 0.9934 and 0.8975 |
| Refinement method | Full-matrix least-squares on F² |
| Data/restraints/parameters | 4961/5/614 |
| Goodness-of-fit on F² | 0.989 |
| Final R indices [I > 2sigma(I)] | R1 = 0.0545, wR2 = 0.0850 |
| R indices (all data) | R1 = 0.0901, wR2 = 0.0988 |
| Absolute structure parameter | −0.01(9) |
| Extinction coefficient | 0.0000(5) |
| Largest diff. Peak and hole | 0.148 and −0.166 e · Å⁻³ |

TABLE 3
Atomic coordinates and equivalent isotropic displacement
parameters (Å²)

| | x | y | z | U(eq) |
|---|---|---|---|---|
| Cl(1) | 2499(1) | 10252(3) | 5466(1) | 96(1) |
| Cl(2) | 1134(1) | 11625(3) | −3072(1) | 116(1) |
| O(25) | 2849(2) | 4866(6) | −1036(2) | 64(1) |
| F(14) | 4242(2) | 6799(5) | −181(2) | 74(1) |
| N(15) | 3383(3) | 3883(7) | 272(2) | 54(1) |
| N(22) | 1327(3) | 1455(7) | −732(2) | 50(1) |
| O(17) | 3199(2) | 1171(5) | 888(2) | 65(1) |
| N(58) | 6088(4) | 10093(8) | 4913(2) | 67(1) |
| O(1) | 5910(2) | 6127(6) | 2485(2) | 58(1) |
| N(26) | 1488(3) | 4750(7) | −1353(2) | 53(1) |
| N(39) | 8696(3) | 2084(7) | 8311(2) | 52(1) |
| N(23) | 2068(3) | 2418(6) | −641(2) | 49(1) |
| C(3) | 6243(4) | 9117(9) | 2921(3) | 59(2) |
| C(62) | 3564(4) | 10138(9) | 5307(4) | 65(2) |
| C(11) | 3783(3) | 4994(8) | 784(3) | 46(1) |
| C(43) | 8301(4) | 6329(8) | 6776(3) | 55(2) |
| C(8) | 4623(3) | 7387(8) | 1691(3) | 49(2) |
| C(12) | 4211(3) | 6501(9) | 538(3) | 52(2) |
| C(27) | 1441(4) | 6387(8) | −1766(3) | 49(2) |
| C(29) | 2031(4) | 8925(9) | −2433(3) | 60(2) |
| N(47) | 8116(4) | 7762(7) | 6289(3) | 65(2) |
| C(45) | 7786(4) | 4345(8) | 7723(3) | 54(2) |
| C(13) | 4633(3) | 7740(8) | 969(3) | 54(2) |
| F(46) | 6921(2) | 6482(6) | 7138(2) | 94(1) |
| C(24) | 2184(4) | 4055(8) | −1025(3) | 47(2) |
| C(61) | 4099(5) | 10090(9) | 5874(3) | 74(2) |
| N(7) | 5114(3) | 8602(6) | 2148(2) | 49(1) |
| C(41) | 9193(4) | 4156(8) | 7373(3) | 59(2) |
| C(59) | 5248(4) | 10071(8) | 5082(3) | 60(2) |
| C(18) | 2733(3) | 1227(8) | −331(3) | 47(1) |
| C(5) | 5439(4) | 11695(9) | 2504(3) | 70(2) |
| C(21) | 1425(3) | −159(9) | −457(3) | 55(2) |
| O(33) | 9509(2) | 4009(6) | 8985(2) | 67(1) |
| C(10) | 3771(3) | 4722(8) | 1513(3) | 51(2) |
| C(64) | 4698(4) | 10081(10) | 4517(3) | 74(2) |
| C(44) | 7685(4) | 5668(9) | 7210(3) | 56(2) |
| O(57) | 6703(3) | 9802(7) | 6016(2) | 84(1) |
| C(20) | 2243(3) | −532(7) | −148(3) | 54(2) |
| N(54) | 7519(4) | 10222(8) | 4291(2) | 69(2) |
| C(31) | 560(4) | 8680(9) | −2310(3) | 73(2) |
| C(63) | 3854(4) | 10110(10) | 4628(3) | 72(2) |
| C(4) | 6099(4) | 10979(9) | 2906(3) | 63(2) |
| C(2) | 5778(3) | 7822(9) | 2520(3) | 44(2) |
| C(38) | 8368(4) | 361(10) | 8177(3) | 67(2) |
| C(60) | 4935(5) | 10087(9) | 5762(3) | 75(2) |
| C(48) | 8620(5) | 8404(10) | 5768(4) | 71(2) |
| C(32) | 667(4) | 7084(9) | −1907(3) | 65(2) |
| C(9) | 4199(3) | 5907(8) | 1963(3) | 55(2) |
| C(16) | 3126(3) | 2087(8) | 347(3) | 48(2) |
| C(6) | 4974(4) | 10502(9) | 2137(3) | 68(2) |
| C(56) | 6753(5) | 9969(10) | 5364(3) | 72(2) |
| C(36) | 8994(4) | −825(9) | 9221(4) | 69(2) |
| C(40) | 8561(4) | 3560(8) | 7799(3) | 49(2) |
| C(42) | 9073(4) | 5552(8) | 6865(3) | 58(2) |
| C(37) | 8504(4) | −1083(10) | 8618(4) | 73(2) |
| C(35) | 9320(3) | 858(9) | 9359(3) | 59(2) |
| C(34) | 9203(3) | 2438(9) | 8903(3) | 55(2) |
| N(55) | 7498(4) | 9932(8) | 5025(2) | 68(2) |
| C(28) | 2115(4) | 7335(9) | −2030(3) | 61(2) |
| C(19) | 3416(3) | 816(8) | −872(3) | 64(2) |
| C(52) | 8865(4) | 10446(12) | 4701(3) | 99(3) |
| C(51) | 8383(5) | 11815(10) | 5864(3) | 108(3) |
| C(30) | 1243(4) | 9570(9) | −2568(3) | 68(2) |
| C(50) | 8331(4) | 10136(10) | 5359(3) | 70(2) |
| C(53) | 8259(5) | 10513(9) | 4111(4) | 81(2) |
| O(49) | 9285(3) | 7707(8) | 5636(2) | 114(2) |

TABLE 4
Bond lengths [Å] and angles [°]

| | |
|---|---|
| Cl(1)—C(62) | 1.754(6) |
| Cl(2)—C(30) | 1.755(6) |
| O(25)—C(24) | 1.223(6) |
| F(14)—C(12) | 1.367(5) |
| N(15)—C(16) | 1.356(6) |
| N(15)—C(11) | 1.404(6) |
| N(22)—C(21) | 1.273(7) |
| N(22)—N(23) | 1.394(5) |
| O(17)—C(16) | 1.214(6) |
| N(58)—C(56) | 1.372(7) |
| N(58)—C(59) | 1.398(7) |
| O(1)—C(2) | 1.231(6) |
| N(26)—C(24) | 1.377(6) |
| N(26)—C(27) | 1.405(7) |
| N(39)—C(38) | 1.364(7) |
| N(39)—C(34) | 1.404(6) |
| N(39)—C(40) | 1.443(6) |
| N(23)—C(24) | 1.387(6) |
| N(23)—C(18) | 1.490(6) |
| C(3)—C(4) | 1.351(7) |
| C(3)—C(2) | 1.410(7) |
| C(62)—C(63) | 1.359(7) |
| C(62)—C(61) | 1.372(7) |
| C(11)—C(12) | 1.361(7) |
| C(11)—C(10) | 1.382(6) |
| C(43)—C(44) | 1.373(7) |
| C(43)—C(42) | 1.378(7) |
| C(43)—N(47) | 1.405(7) |
| C(8)—C(9) | 1.362(7) |
| C(8)—C(13) | 1.379(7) |
| C(8)—N(7) | 1.455(6) |
| C(12)—C(13) | 1.380(7) |
| C(27)—C(32) | 1.375(7) |
| C(27)—C(28) | 1.377(7) |
| C(29)—C(28) | 1.372(7) |
| C(29)—C(30) | 1.379(7) |
| N(47)—C(48) | 1.355(8) |
| C(45)—C(44) | 1.359(7) |
| C(45)—C(40) | 1.382(7) |
| F(46)—C(44) | 1.374(6) |
| C(61)—C(60) | 1.371(7) |
| N(7)—C(6) | 1.376(7) |
| N(7)—C(2) | 1.398(6) |
| C(41)—C(40) | 1.368(7) |
| C(41)—C(42) | 1.393(7) |
| C(59)—C(60) | 1.374(7) |
| C(59)—C(64) | 1.384(7) |
| C(18)—C(20) | 1.526(7) |
| C(18)—C(19) | 1.532(6) |
| C(18)—C(16) | 1.548(7) |
| C(5)—C(6) | 1.329(7) |
| C(5)—C(4) | 1.404(7) |
| C(21)—C(20) | 1.470(7) |
| O(33)—C(34) | 1.237(6) |
| C(10)—C(9) | 1.382(6) |
| C(64)—C(63) | 1.383(7) |
| O(57)—C(56) | 1.233(6) |
| N(54)—C(53) | 1.265(7) |
| N(54)—N(55) | 1.394(6) |
| C(31)—C(30) | 1.366(7) |
| C(31)—C(32) | 1.379(7) |
| C(38)—C(37) | 1.341(8) |
| C(48)—O(49) | 1.212(7) |
| C(48)—C(50) | 1.530(9) |
| C(56)—N(55) | 1.367(7) |
| C(36)—C(35) | 1.338(7) |
| C(36)—C(37) | 1.394(8) |
| C(35)—C(34) | 1.429(7) |
| N(55)—C(50) | 1.494(7) |
| C(52)—C(53) | 1.479(7) |
| C(52)—C(50) | 1.526(8) |
| C(51)—C(50) | 1.531(8) |
| C(16)—N(15)—C(11) | 127.3(5) |
| C(21)—N(22)—N(23) | 106.9(4) |
| C(56)—N(58)—C(59) | 128.5(6) |
| C(24)—N(26)—C(27) | 126.2(5) |
| C(38)—N(39)—C(34) | 122.3(5) |
| C(38)—N(39)—C(40) | 118.6(5) |
| C(34)—N(39)—C(40) | 118.9(5) |
| C(24)—N(23)—N(22) | 118.0(4) |
| C(24)—N(23)—C(18) | 125.9(4) |
| N(22)—N(23)—C(18) | 112.8(4) |
| C(4)—C(3)—C(2) | 122.9(6) |
| C(63)—C(62)—C(61) | 120.5(6) |
| C(63)—C(62)—Cl(1) | 120.2(6) |
| C(61)—C(62)—Cl(1) | 119.3(5) |
| C(12)—C(11)—C(10) | 117.1(5) |
| C(12)—C(11)—N(15) | 116.7(5) |
| C(10)—C(11)—N(15) | 126.2(5) |
| C(44)—C(43)—C(42) | 116.8(6) |
| C(44)—C(43)—N(47) | 118.8(6) |
| C(42)—C(43)—N(47) | 124.4(6) |
| C(9)—C(8)—C(13) | 121.2(5) |
| C(9)—C(8)—N(7) | 121.2(5) |
| C(13)—C(8)—N(7) | 117.6(5) |
| C(11)—C(12)—F(14) | 118.6(5) |
| C(11)—C(12)—C(13) | 124.0(5) |
| F(14)—C(12)—C(13) | 117.4(5) |
| C(32)—C(27)—C(28) | 118.4(6) |
| C(32)—C(27)—N(26) | 117.1(5) |
| C(28)—C(27)—N(26) | 124.4(6) |
| C(28)—C(29)—C(30) | 118.0(6) |
| C(48)—N(47)—C(43) | 126.0(6) |
| C(44)—C(45)—C(40) | 117.6(6) |
| C(8)—C(13)—C(12) | 116.9(5) |
| O(25)—C(24)—N(26) | 122.9(5) |
| O(25)—C(24)—N(23) | 121.9(5) |
| N(26)—C(24)—N(23) | 115.2(5) |
| C(60)—C(61)—C(62) | 120.2(6) |
| C(6)—N(7)—C(2) | 121.8(5) |
| C(6)—N(7)—C(8) | 119.3(5) |
| C(2)—N(7)—C(8) | 118.4(4) |
| C(40)—C(41)—C(42) | 121.3(6) |
| C(60)—C(59)—C(64) | 118.3(6) |
| C(60)—C(59)—N(58) | 124.8(6) |
| C(64)—C(59)—N(58) | 116.9(5) |
| N(23)—C(18)—C(20) | 100.5(4) |
| N(23)—C(18)—C(19) | 111.9(4) |
| C(20)—C(18)—C(19) | 111.6(5) |
| N(23)—C(18)—C(16) | 112.9(4) |
| C(20)—C(18)—C(16) | 110.8(5) |
| C(19)—C(18)—C(16) | 109.0(4) |
| C(6)—C(5)—C(4) | 118.4(6) |
| N(22)—C(21)—C(20) | 115.9(5) |
| C(11)—C(10)—C(9) | 120.8(5) |
| C(63)—C(64)—C(59) | 121.2(6) |
| C(45)—C(44)—F(46) | 118.2(6) |
| C(45)—C(44)—C(43) | 124.9(6) |
| F(46)—C(44)—C(43) | 116.9(6) |
| C(21)—C(20)—C(18) | 103.3(5) |
| C(53)—N(54)—N(55) | 108.2(6) |
| C(30)—C(31)—C(32) | 118.6(6) |
| C(62)—C(63)—C(64) | 119.1(6) |
| C(3)—C(4)—C(5) | 120.1(6) |
| O(1)—C(2)—N(7) | 119.9(5) |
| O(1)—C(2)—C(3) | 125.6(6) |
| N(7)—C(2)—C(3) | 114.5(5) |
| C(37)—C(38)—N(39) | 121.2(6) |
| C(61)—C(60)—C(59) | 120.6(6) |
| O(49)—C(48)—N(47) | 123.0(7) |
| O(49)—C(48)—C(50) | 120.1(7) |
| N(47)—C(48)—C(50) | 116.8(6) |
| C(27)—C(32)—C(31) | 121.2(6) |
| C(8)—C(9)—C(10) | 119.9(5) |
| O(17)—C(16)—N(15) | 124.5(5) |
| O(17)—C(16)—C(18) | 120.9(5) |
| N(15)—C(16)—C(18) | 114.6(5) |
| C(5)—C(6)—N(7) | 122.1(6) |
| O(57)—C(56)—N(55) | 121.4(6) |
| O(57)—C(56)—N(58) | 124.5(7) |
| N(55)—C(56)—N(58) | 114.0(6) |
| C(35)—C(36)—C(37) | 120.0(6) |
| C(41)—C(40)—C(45) | 119.6(6) |
| C(41)—C(40)—N(39) | 120.3(5) |
| C(45)—C(40)—N(39) | 120.1(5) |
| C(43)—C(42)—C(41) | 119.8(6) |
| C(38)—C(37)—C(36) | 119.5(6) |
| C(36)—C(35)—C(34) | 122.8(6) |
| O(33)—C(34)—N(39) | 119.7(6) |
| O(33)—C(34)—C(35) | 126.1(6) |
| N(39)—C(34)—C(35) | 114.1(6) |
| C(56)—N(55)—N(54) | 118.8(5) |
| C(56)—N(55)—C(50) | 126.9(5) |
| N(54)—N(55)—C(50) | 112.2(5) |
| C(29)—C(28)—C(27) | 121.8(6) |
| C(53)—C(52)—C(50) | 103.5(5) |
| C(31)—C(30)—C(29) | 121.9(6) |
| C(31)—C(30)—Cl(2) | 119.9(5) |
| C(29)—C(30)—Cl(2) | 118.1(5) |
| N(55)—C(50)—C(52) | 100.8(5) |
| N(55)—C(50)—C(48) | 114.1(6) |
| C(52)—C(50)—C(48) | 110.5(6) |
| N(55)—C(50)—C(51) | 112.7(6) |
| C(52)—C(50)—C(51) | 110.9(6) |
| C(48)—C(50)—C(51) | 107.8(5) |
| N(54)—C(53)—C(52) | 115.1(6) |

TABLE 5
Anisotropic displacement parameters (Å² × 10³)

| | U¹¹ | U²² | U³³ | U²³ | U¹³ | U¹² |
|---|---|---|---|---|---|---|
| Cl(1) | 113(2) | 74(1) | 99(1) | 1(1) | 18(1) | 1(1) |
| Cl(2) | 119(2) | 89(2) | 138(2) | 61(1) | 43(1) | 40(1) |
| O(25) | 52(2) | 70(3) | 70(3) | 26(2) | −19(2) | −27(2) |
| F(14) | 104(3) | 64(2) | 53(2) | 9(2) | −16(2) | −24(2) |
| N(15) | 69(3) | 41(3) | 52(3) | 4(3) | −17(3) | −11(3) |
| N(22) | 47(3) | 47(3) | 57(3) | 0(3) | −6(2) | −11(3) |
| O(17) | 98(3) | 42(3) | 56(3) | 9(2) | −12(2) | −6(2) |
| N(58) | 91(4) | 65(4) | 45(3) | 0(3) | −5(3) | 13(4) |
| O(1) | 77(3) | 42(3) | 55(2) | −7(2) | −15(2) | 8(2) |
| N(26) | 57(3) | 51(3) | 49(3) | 6(3) | −13(2) | −9(3) |
| N(39) | 51(3) | 49(4) | 58(3) | 0(3) | −3(2) | −12(3) |
| N(23) | 45(3) | 48(3) | 53(3) | 8(3) | −7(2) | −14(3) |
| C(3) | 67(5) | 64(5) | 46(4) | −1(3) | −8(3) | −14(4) |
| C(62) | 85(5) | 40(4) | 70(4) | 0(4) | 7(4) | 11(4) |
| C(11) | 49(4) | 39(4) | 49(3) | 3(3) | −12(3) | −6(3) |
| C(43) | 69(4) | 52(4) | 44(4) | −1(3) | −3(3) | 4(4) |
| C(8) | 51(4) | 36(4) | 59(4) | −4(3) | 0(3) | 8(3) |
| C(12) | 57(4) | 52(4) | 46(4) | 5(3) | −9(3) | 1(3) |
| C(27) | 64(4) | 47(4) | 35(3) | 3(3) | −8(3) | −9(3) |
| C(29) | 62(4) | 57(4) | 60(4) | 12(4) | 18(3) | 0(4) |
| N(47) | 86(5) | 63(4) | 44(3) | 0(3) | −10(3) | 11(3) |
| C(45) | 56(4) | 58(4) | 49(4) | 1(3) | 0(3) | 0(3) |
| C(13) | 51(4) | 44(4) | 66(4) | 14(3) | −10(3) | −8(3) |
| F(46) | 80(3) | 119(4) | 84(2) | 25(2) | 7(2) | 37(3) |
| C(24) | 50(4) | 55(4) | 35(3) | 3(3) | −4(3) | −4(3) |
| C(61) | 119(6) | 52(4) | 49(4) | 0(4) | 4(4) | 4(5) |
| N(7) | 60(3) | 34(3) | 53(3) | −5(3) | −8(2) | 3(3) |
| C(41) | 52(4) | 61(5) | 65(4) | −8(4) | 1(3) | 8(3) |
| C(59) | 98(6) | 40(4) | 40(4) | −2(3) | −2(4) | 3(4) |
| C(18) | 44(4) | 40(4) | 57(4) | 1(3) | −6(3) | −5(3) |
| C(5) | 83(5) | 37(4) | 91(5) | −13(4) | −8(4) | −10(4) |
| C(21) | 54(4) | 49(4) | 62(4) | −10(4) | 0(3) | −16(3) |
| O(33) | 79(3) | 56(3) | 67(3) | −1(2) | −14(2) | −8(2) |
| C(10) | 54(4) | 39(4) | 58(4) | 5(3) | −6(3) | −5(3) |
| C(64) | 97(5) | 71(5) | 53(4) | −2(4) | −5(4) | −3(5) |
| C(44) | 56(4) | 62(5) | 49(4) | −3(3) | −7(3) | 19(4) |
| O(57) | 114(4) | 89(4) | 48(3) | 4(3) | −12(2) | 26(3) |
| C(20) | 56(4) | 43(4) | 63(4) | 0(3) | −3(3) | −1(3) |
| N(54) | 101(4) | 60(4) | 46(3) | −7(3) | −8(3) | −1(4) |
| C(31) | 64(5) | 69(5) | 87(5) | 25(4) | −4(4) | 12(4) |
| C(63) | 92(5) | 64(5) | 60(4) | 3(4) | −8(4) | 11(5) |
| C(4) | 73(5) | 53(5) | 62(4) | −6(3) | 3(4) | −20(4) |
| C(2) | 47(4) | 43(4) | 41(3) | −10(3) | −6(3) | −4(3) |
| C(38) | 73(5) | 59(5) | 68(4) | 3(4) | −4(3) | −7(4) |
| C(60) | 106(6) | 60(5) | 60(4) | −2(4) | −10(4) | 17(5) |
| C(48) | 77(5) | 77(5) | 58(5) | 5(4) | 0(4) | 9(4) |
| C(32) | 58(4) | 61(5) | 74(4) | 16(4) | −9(3) | −14(4) |
| C(9) | 62(4) | 51(4) | 51(4) | 0(3) | −10(3) | −4(3) |
| C(16) | 52(4) | 41(4) | 52(4) | 3(3) | −7(3) | −1(3) |
| C(6) | 76(5) | 45(5) | 83(5) | 3(4) | −15(4) | 10(4) |
| C(56) | 108(6) | 59(5) | 49(4) | 6(4) | −19(4) | 15(5) |
| C(36) | 63(5) | 51(5) | 92(5) | 24(4) | 16(4) | −1(4) |
| C(40) | 57(4) | 38(4) | 52(4) | −10(3) | 0(3) | 1(3) |
| C(42) | 58(4) | 62(5) | 54(4) | 13(3) | −5(3) | −2(3) |
| C(37) | 78(5) | 51(5) | 91(5) | −2(4) | −5(4) | −21(4) |
| C(35) | 59(4) | 61(5) | 58(4) | 18(4) | −1(3) | 4(4) |
| C(34) | 55(4) | 53(5) | 56(4) | −3(4) | 3(3) | −5(4) |
| N(55) | 101(4) | 62(4) | 41(3) | −8(3) | −8(3) | 7(4) |
| C(28) | 57(4) | 63(5) | 61(4) | 8(4) | 8(3) | −1(4) |
| C(19) | 61(4) | 65(5) | 64(4) | −8(3) | 4(3) | −5(3) |
| C(52) | 106(6) | 111(7) | 79(5) | 37(5) | −23(4) | −28(5) |
| C(51) | 175(8) | 66(5) | 83(5) | −1(5) | −51(5) | 1(5) |
| C(30) | 81(5) | 63(5) | 60(4) | 16(3) | 16(4) | 10(4) |
| C(50) | 97(5) | 64(5) | 51(4) | 7(4) | −25(4) | 1(4) |
| C(53) | 110(6) | 65(5) | 69(5) | 9(4) | 4(5) | −4(5) |
| O(49) | 105(4) | 133(5) | 104(4) | 55(3) | 27(3) | 38(4) |

TABLE 6
Hydrogen coordinates (×10⁴) and isotropic displacement
parameters (A² × 10³)

| | x | y | z | U(eq) |
|---|---|---|---|---|
| H(3) | 6668 | 8670 | 3208 | 71 |
| H(29) | 2490 | 9549 | −2610 | 72 |
| H(45) | 7350 | 3980 | 8012 | 65 |
| H(13) | 4912 | 8765 | 782 | 64 |
| H(61) | 3894 | 10059 | 6337 | 88 |
| H(41) | 9713 | 3618 | 7423 | 71 |
| H(5) | 5331 | 12974 | 2493 | 84 |
| H(21) | 1005 | −1046 | −451 | 66 |
| H(10) | 3471 | 3730 | 1702 | 61 |
| H(64) | 4899 | 10068 | 4053 | 88 |
| H(20^{a}) | 2491 | −1635 | −360 | 65 |
| H(20B) | 2209 | −707 | 363 | 65 |
| H(31) | 34 | 9141 | −2405 | 88 |
| H(63) | 3491 | 10111 | 4244 | 86 |
| H(4) | 6437 | 11788 | 3163 | 75 |
| H(38) | 8044 | 184 | 7773 | 80 |
| H(60) | 5294 | 10096 | 6149 | 91 |
| H(32) | 206 | 6468 | −1728 | 78 |
| H(9) | 4196 | 5693 | 2452 | 66 |
| H(6) | 4540 | 10966 | 1865 | 82 |
| H(36) | 9095 | −1823 | 9527 | 82 |
| H(42) | 9513 | 5958 | 6587 | 69 |
| H(37) | 8273 | −2248 | 8523 | 88 |
| H(35) | 9635 | 1006 | 9770 | 71 |
| H(28) | 2642 | 6885 | −1931 | 73 |
| H(19^{a}) | 3202 | 27 | −1243 | 95 |
| H(19B) | 3866 | 193 | −638 | 95 |
| H(19C) | 3608 | 1970 | −1075 | 95 |
| H(52^{a}) | 9170 | 11612 | 4735 | 119 |
| H(52B) | 9252 | 9424 | 4636 | 119 |
| H(51^{a}) | 8288 | 12948 | 5601 | 162 |
| H(51B) | 8922 | 11857 | 6078 | 162 |
| H(51C) | 7973 | 11692 | 6229 | 162 |
| H(53) | 8412 | 10751 | 3642 | 97 |
| H(15) | 3280(30) | 4570(60) | −189(13) | 53(15) |
| H(26) | 1050(20) | 3770(50) | −1280(30) | 73(19) |
| H(58) | 6320(30) | 10100(100) | 4415(12) | 110(20) |
| H(47) | 7553(19) | 8360(90) | 6280(30) | 120(30) |

TABLE 7
Torsion angles [°]

| | |
|---|---|
| C(21)-N(22)-N(23)-C(24) | 166.5(5) |
| C(21)-N(22)-N(23)-C(18) | 5.7(6) |
| C(16)-N(15)-C(11)-C(12) | 159.9(5) |
| C(16)-N(15)-C(11)-C(10) | −21.4(9) |
| C(10)-C(11)-C(12)-F(14) | 179.4(5) |
| N(15)-C(11)-C(12)-F(14) | −1.8(7) |
| C(10)-C(11)-C(12)-C(13) | 0.7(8) |
| N(15)-C(11)-C(12)-C(13) | 179.6(5) |
| C(24)-N(26)-C(27)-C(32) | −165.0(5) |
| C(24)-N(26)-C(27)-C(28) | 14.4(8) |
| C(44)-C(43)-N(47)-C(48) | −171.4(6) |
| C(42)-C(43)-N(47)-C(48) | 10.2(9) |
| C(9)-C(8)-C(13)-C(12) | −1.5(8) |
| N(7)-C(8)-C(13)-C(12) | 175.7(5) |
| C(11)-C(12)-C(13)-C(8) | 1.0(9) |
| F(14)-C(12)-C(13)-C(8) | −177.7(4) |
| C(27)-N(26)-C(24)-O(25) | 3.2(9) |
| C(27)-N(26)-C(24)-N(23) | 179.5(5) |
| N(22)-N(23)-C(24)-O(25) | −171.7(5) |
| C(18)-N(23)-C(24)-O(25) | −13.7(8) |
| N(22)-N(23)-C(24)-N(26) | 12.0(7) |
| C(18)-N(23)-C(24)-N(26) | 170.0(4) |
| C(63)-C(62)-C(61)-C(60) | 2.5(10) |
| Cl(1)-C(62)-C(61)-C(60) | −177.0(5) |
| C(9)-C(8)-N(7)-C(6) | −125.6(6) |
| C(13)-C(8)-N(7)-C(6) | 57.1(7) |
| C(9)-C(8)-N(7)-C(2) | 62.0(7) |
| C(13)-C(8)-N(7)-C(2) | −115.3(6) |
| C(56)-N(58)-C(59)-C(60) | 6.3(11) |
| C(56)-N(58)-C(59)-C(64) | −175.4(6) |
| C(24)-N(23)-C(18)-C(20) | −166.9(5) |
| N(22)-N(23)-C(18)-C(20) | −7.9(5) |
| C(24)-N(23)-C(18)-C(19) | −48.3(7) |
| N(22)-N(23)-C(18)-C(19) | 110.7(5) |
| C(24)-N(23)-C(18)-C(16) | 75.1(6) |
| N(22)-N(23)-C(18)-C(16) | −125.9(4) |
| N(23)-N(22)-C(21)-C(20) | −0.6(6) |
| C(12)-C(11)-C(10)-C(9) | −2.0(8) |
| N(15)-C(11)-C(10)-C(9) | 179.3(5) |
| C(60)-C(59)-C(64)-C(63) | 0.1(10) |
| N(58)-C(59)-C(64)-C(63) | −178.3(6) |
| C(40)-C(45)-C(44)-F(46) | −178.9(5) |
| C(40)-C(45)-C(44)-C(43) | −2.2(8) |
| C(42)-C(43)-C(44)-C(45) | 1.3(8) |
| N(47)-C(43)-C(44)-C(45) | −177.3(5) |
| C(42)-C(43)-C(44)-F(46) | 178.1(5) |
| N(47)-C(43)-C(44)-F(46) | −0.5(7) |
| N(22)-C(21)-C(20)-C(18) | −4.3(6) |
| N(23)-C(18)-C(20)-C(21) | 6.7(5) |
| C(19)-C(18)-C(20)-C(21) | −112.1(5) |
| C(16)-C(18)-C(20)-C(21) | 126.3(5) |
| C(61)-C(62)-C(63)-C(64) | −1.6(10) |
| Cl(1)-C(62)-C(63)-C(64) | 177.9(5) |
| C(59)-C(64)-C(63)-C(62) | 0.3(10) |
| C(2)-C(3)-C(4)-C(5) | 2.8(10) |
| C(6)-C(5)-C(4)-C(3) | −0.6(9) |
| C(6)-N(7)-C(2)-O(1) | −175.9(5) |
| C(8)-N(7)-C(2)-O(1) | −3.8(8) |
| C(6)-N(7)-C(2)-C(3) | 4.8(8) |
| C(8)-N(7)-C(2)-C(3) | 176.9(5) |
| C(4)-C(3)-C(2)-O(1) | 176.0(6) |
| C(4)-C(3)-C(2)-N(7) | −4.7(8) |
| C(34)-N(39)-C(38)-C(37) | 1.2(9) |
| C(40)-N(39)-C(38)-C(37) | 177.0(6) |
| C(62)-C(61)-C(60)-C(59) | −2.1(10) |
| C(64)-C(59)-C(60)-C(61) | 0.8(10) |
| N(58)-C(59)-C(60)-C(61) | 179.1(6) |
| C(43)-N(47)-C(48)-O(49) | 5.0(10) |
| C(43)-N(47)-C(48)-C(50) | −172.7(5) |
| C(28)-C(27)-C(32)-C(31) | 0.8(8) |
| N(26)-C(27)-C(32)-C(31) | −179.8(5) |
| C(30)-C(31)-C(32)-C(27) | −0.2(9) |
| C(13)-C(8)-C(9)-C(10) | 0.4(8) |
| N(7)-C(8)-C(9)-C(10) | −176.8(5) |
| C(11)-C(10)-C(9)-C(8) | 1.5(8) |
| C(11)-N(15)-C(16)-O(17) | 1.1(9) |
| C(11)-N(15)-C(16)-C(18) | −177.7(5) |
| N(23)-C(18)-C(16)-O(17) | 129.6(5) |
| C(20)-C(18)-C(16)-O(17) | 17.8(7) |
| C(19)-C(18)-C(16)-O(17) | −105.4(6) |
| N(23)-C(18)-C(16)-N(15) | −51.6(6) |
| C(20)-C(18)-C(16)-N(15) | −163.4(5) |
| C(19)-C(18)-C(16)-N(15) | 73.4(6) |
| C(4)-C(5)-C(6)-N(7) | 0.7(9) |
| C(2)-N(7)-C(6)-C(5) | −3.0(9) |
| C(8)-N(7)-C(6)-C(5) | −175.1(5) |
| C(59)-N(58)-C(56)-O(57) | 0.4(12) |
| C(59)-N(58)-C(56)-N(55) | 176.7(6) |
| C(42)-C(41)-C(40)-C(45) | 0.4(8) |
| C(42)-C(41)-C(40)-N(39) | 178.7(5) |
| C(44)-C(45)-C(40)-C(41) | 1.3(8) |
| C(44)-C(45)-C(40)-N(39) | −177.0(5) |
| C(38)-N(39)-C(40)-C(41) | −107.2(6) |
| C(34)-N(39)-C(40)-C(41) | 68.8(6) |
| C(38)-N(39)-C(40)-C(45) | 71.0(7) |
| C(34)-N(39)-C(40)-C(45) | −113.0(6) |
| C(44)-C(43)-C(42)-C(41) | 0.5(8) |
| N(47)-C(43)-C(42)-C(41) | 179.0(5) |
| C(40)-C(41)-C(42)-C(43) | −1.3(8) |
| N(39)-C(38)-C(37)-C(36) | −0.4(9) |
| C(35)-C(36)-C(37)-C(38) | 0.5(10) |
| C(37)-C(36)-C(35)-C(34) | −1.4(10) |
| C(38)-N(39)-C(34)-O(33) | 177.4(5) |
| C(40)-N(39)-C(34)-O(33) | 1.6(8) |
| C(38)-N(39)-C(34)-C(35) | −1.8(7) |
| C(40)-N(39)-C(34)-C(35) | −177.7(5) |
| C(36)-C(35)-C(34)-O(33) | −177.2(6) |
| C(36)-C(35)-C(34)-N(39) | 2.0(8) |
| O(57)-C(56)-N(55)-N(54) | −176.8(6) |
| N(58)-C(56)-N(55)-N(54) | 6.8(9) |
| O(57)-C(56)-N(55)-C(50) | −15.0(11) |
| N(58)-C(56)-N(55)-C(50) | 168.6(6) |
| C(53)-N(54)-N(55)-C(56) | 166.8(6) |
| C(53)-N(54)-N(55)-C(50) | 2.4(7) |
| C(30)-C(29)-C(28)-C(27) | 0.4(9) |
| C(32)-C(27)-C(28)-C(29) | −0.9(8) |
| N(26)-C(27)-C(28)-C(29) | 179.7(5) |
| C(32)-C(31)-C(30)-C(29) | −0.4(10) |
| C(32)-C(31)-C(30)-Cl(2) | −179.0(4) |
| C(28)-C(29)-C(30)-C(31) | 0.3(9) |
| C(28)-C(29)-C(30)-Cl(2) | 179.0(4) |
| C(56)-N(55)-C(50)-C(52) | −166.9(6) |
| N(54)-N(55)-C(50)-C(52) | −4.1(7) |
| C(56)-N(55)-C(50)-C(48) | 74.6(9) |
| N(54)-N(55)-C(50)-C(48) | −122.6(6) |
| C(56)-N(55)-C(50)-C(51) | −48.7(9) |
| N(54)-N(55)-C(50)-C(51) | 114.1(6) |
| C(53)-C(52)-C(50)-N(55) | 4.0(7) |
| C(53)-C(52)-C(50)-C(48) | 125.0(6) |
| C(53)-C(52)-C(50)-C(51) | −115.5(6) |
| O(49)-C(48)-C(50)-N(55) | 126.6(7) |
| N(47)-C(48)-C(50)-N(55) | −55.6(8) |
| O(49)-C(48)-C(50)-C(52) | 13.9(9) |
| N(47)-C(48)-C(50)-C(52) | −168.4(6) |
| O(49)-C(48)-C(50)-C(51) | −107.5(7) |
| N(47)-C(48)-C(50)-C(51) | 70.3(7) |
| N(55)-N(54)-C(53)-C(52) | 0.6(9) |
| C(50)-C(52)-C(53)-N(54) | −3.2(9) |

Symmetry transformations used to generate equivalent atoms:

TABLE 8
Hydrogen bonds [Å and °].

| D-H . . . A | d(D-H) | d(H . . . A) | d(D . . . A) | <(DHA) |
|---|---|---|---|---|
| N(47)—H(47) . . . O(57) | 1.009(2) | 1.79(2) | 2.761(7) | 161(6) |
| N(58)—H(58) . . . N(54) | 1.009(2) | 1.96(5) | 2.600(8) | 119(4) |
| N(26)—H(26) . . . O(33)#1 | 1.009(2) | 2.55(4) | 3.311(6) | 132(4) |
| N(26)—H(26) . . . N(22) | 1.009(2) | 2.00(4) | 2.640(7) | 119(4) |
| N(15)—H(15) . . . O(25) | 1.009(2) | 1.75(2) | 2.695(5) | 154(4) |

Symmetry transformations used to generate equivalent atoms: #1 x − 1, y, z − 1

TABLE 9
Calculated PXRD Peak List

| | Relative |
|---|---|
| Degrees 2θ | Intensity % |
| 5.4 | 11.0 |
| 7.2 | 21.5 |
| 9.4 | 26.7 |
| 10.9 | 67.3 |
| 13.6 | 32.0 |
| 14.2 | 43.6 |
| 14.4 | 58.2 |
| 15.6 | 31.9 |
| 16.4 | 55.4 |
| 16.6 | 46.8 |
| 17.2 | 50.4 |
| 18.9 | 67.5 |
| 19.0 | 45.3 |
| 19.6 | 100.0 |
| 20.6 | 19.0 |
| 21.2 | 49.5 |
| 21.7 | 92.8 |
| 22.7 | 16.7 |
| 23.3 | 57.0 |
| 24.9 | 36.7 |
| 25.2 | 31.6 |
| 25.4 | 29.7 |
| 25.5 | 11.5 |
| 25.7 | 20.6 |
| 26.0 | 31.1 |
| 26.7 | 18.2 |
| 27.0 | 21.3 |
| 27.2 | 15.8 |
| 27.4 | 23.8 |
| 28.8 | 14.8 |
| 29.0 | 23.3 |

### EXAMPLES

### Example 1

(R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide}

Step 1. Synthesis of 1-(4-Amino-3-fluoro-phenyl)-1H-pyridin-2-one (3).

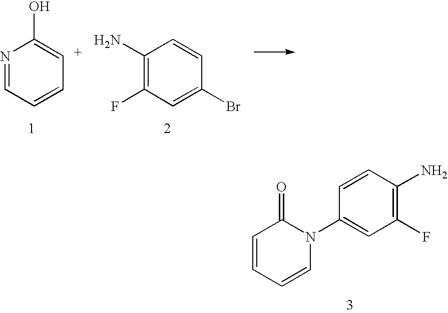

In a three liter four necked flask equipped with a mechanical stirrer and reflux condenser, was charged, under nitrogen, a mixture of 4-bromo-2-fluoroaniline (2) (510 g, 2.684 mol), 2-hydroxypyridine (1) (280 g, 2.949 mol), powdered potassium carbonate (221 g, 1.602 mol), and copper (I) iodide (25 g, 0.131 mol) in dimethylformamide (1.5 L). The mixture was stirred with heating at 125-130° C. (gentle reflux) for 22 hr. Progress of the reaction was followed by TLC. Approximately 800 mL of solvent was distilled off in vacuo. The thick residue was cooled to room temperature and 15% NH₄OH (1L) was added with vigorous stirring. Water (1.5 L) was added and the resulting suspension was stirred at 10-15° C. (ice-bath) for 1 hr. & filtered. The solid was washed with water (1.5 L) and pressed dry under suction. Further drying in vacuo at 35° C. for 18 hrs. afforded 404 g (73.8%) of product (3). Additional product was obtained from the filtrate.

¹H NMR (DMSO) ppm: 7.55 (d, 1H), 7.4 (t, 1H), 7.05 (2d, 1H), 6.75-6.9 (m, 2H), 5.35 (s, 2H).

Step 2. Synthesis of N-[2-Fluoro-4-(2-oxo-2H-pyridin-1-yl)-phenyl]-2-methyl-acrylamide (5).

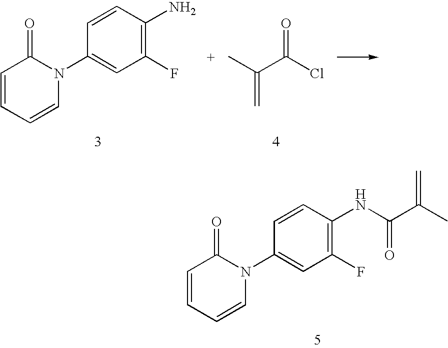

Procedure: To a 10 L reactor was added a mixture of 1-(4-Amino-3-fluoro-phenyl)-1H-pyridin-2-one (3) (363 g, 1.77 mol) in tetrahydrofuran (3.75 L). The mechanically stirred mixture was cooled to 0° with an ice-acetone bath and a solution of potassium carbonate (450 g, 3.27 mol) in water (2.25 L) was added. Methacryloyl chloride (4) (370 g, 3.55 mol) was added in a stream over a 2 hr. period, with the temperature<10 degrees C. Toward the end of the addition most of the solid had gone into solution. The resulting mixture was stirred at 0° C. for 3 hrs. During this time, a solid separated. The solid was filtered off and pressed dry under suction. The filtrate was concentrated in vacuo to a thick slurry. The slurry was filtered and the solid was pressed dry under suction. The two solids were combined and stirred with dichloromethane (2.5 L) at 30° C. until a solution formed. The solution was cooled to room temperature and stirred vigorously with brine (1 L). The layers were separated and the organic layer was dried over magnesium sulfate, filtered and concentrated to approximately 1.5 L. The resulting suspension was stirred at 5° C. over a weekend and filtered. The solid was washed with cold (5° C.) dichloromethane (100 mL), then with diethyl ether (250 mL) and pressed dry under suction. Further drying in vacuo at 35° C. for 18 hr afforded 318 g (66%) of product (5). Additional material of lesser purity was obtained from the filtrate.

¹H NMR (DMSO) ppm: 9.7 (s, 1H), 7.6-7.65 (m, 2H), 7.4-7.5 (m, 2H), 7.2 (2d, 1H), 6.45 (d, 1H), 6.25 (t, 1H), 5.85 (s, 1H), 5.5 (s, 1H), 1.9 (s, 3H).

Step 3. Synthesis of 3-Methyl-3,4-dihydro-2H-pyrazole-3-carboxylic acid [2-fluoro-4-(2-oxo-2H-pyridin-1-yl)-phenyl]-amide (6).

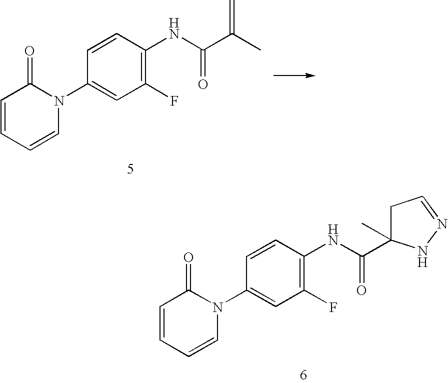

Procedure: To a 12 L flask equipped with a mechanical stirrer, a pressure equalized 2000 mL addition funnel, and a thermometer was charged, under nitrogen, (5) (387 g, 1.42 mol) and methylene chloride (8.0 L). To the stirred solution at 20-21° C. was added 2M trimethylsilyl diazomethane (1000 mL, 2.0 mol, 1.4 equivalents.) in diethyl ether over a 75-minute period. The resulting mixture was stirred at 21-22° C. for 18 hr. MS analysis of the reaction mixture indicated the presence of a mixture of the TMS-pyrazole intermediate (M⁺=386) and (5). To the mixture was added another aliquot of 2M trimethylsilyl diazomethane in diethyl ether (423 mL, 0.846 mol, 0.6 equivalents.) over a 30-minute period. Stirring was continued at 21-22° C. for another 22 hr. MS analysis of the reaction mixture indicated the presence of the desired TMS-pyrazole intermediate (M⁺=386) and a small amount of (5) (M⁺=272). The solution was cooled to 0° C. and trifluoroacetic acid (345 mL, 2.0 mol, 1.4 equivalents.) was added over a 1.5 hr. period while maintaining the temperature below 8° C. during the addition. The solution was stirred at 0-5° C. for 3 hr. Triethylamine (1400 mL, 1016 g, 10 mol) was added over a 1.5 hr. period while maintaining the temperature below 10° C. The resulting mixture was concentrated in vacuo to a thick gum. The thick product mixture was taken up in ethyl acetate (2 L). A solution formed. The solution was seeded and left standing overnight at 5° C. The resulting suspension was stirred at −10° C. for 2 hr. The suspension was filtered. The filter cake was washed with cold ethyl acetate (200 mL). The filter cake was pressed dry under suction. Further drying in vacuo at 35° C. afforded 173 g (39%) of the product as the first crop. The filtrate was washed with water (1×1000 mL, 1×500 mL). The organic phase was dried with MgSO₄. The product solution was filtered and the drying agent discarded. The organic phase was concentrated in vacuo to dryness. The residual oil was taken up in ethyl acetate (250 mL). The suspension was stirred at −10° C. for 2 hr. This suspension was filtered. The filter cake was rinsed with cold ethyl acetate (40 mL). The filtered cake was pressed dry under suction. Drying the filter cake in vacuo at 35° C. afforded 60.5 g (13.5%) of product. The combined water washings were extracted with methylene chloride (1×1000 mL, 1×500 mL). The combined extracts were dried with MgSO₄. The product solution was filtered and the drying agent discarded. The organic phase was concentrated in vacuo to dryness. The residual oil was taken up in ethyl acetate (800 mL). The suspension was stirred at −10° C. for 2 hr. This suspension was filtered. The filter cake was rinsed with cold ethyl acetate (100 mL). The filtered cake was pressed dry under suction in vacuo at 35° C. to afforded 125 g (28%) of product (6). Total product obtained 173+61+125=359 g (80%). HPLC analysis of the combined product: 95% (area/area).

¹H NMR (DMSO) ppm: 9.65 (s, 1H), 8.05 (t, 1H), 7.65 (d, 1H), 7.45-7.55 (m, 2H), 7.25 (d, 1H), 7.2 (s, 1H), 6.75 (s, 1H), 6.45 (d, 1H), 6.3 (t, 1H), 2.7-2.95 (2d, 2H), 1.35 (s, 3H).

Step 4. Synthesis of 5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide} (8)

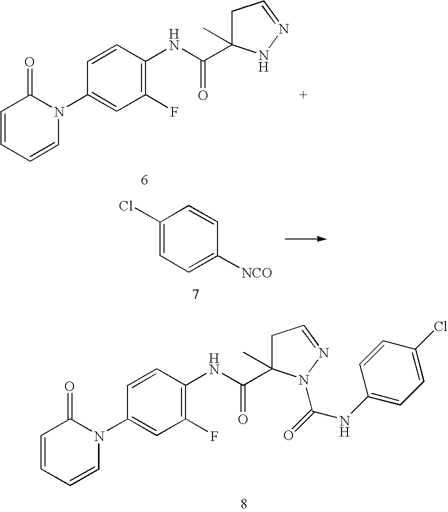

Procedure: To a stirred mixture of 3-methyl-3,4-dihydro-2H-pyrazole-3-carboxylic acid [2-fluoro-4-(2-oxo-2H-pyridin-1-yl)-phenyl]-amide (6) (152.5 g, 0.486 mol), 4-chlorophenylisocyanate (7) (82.1 g, 0.535 mol), and tetrahydrofuran (3.5 L) was added dropwise over a 40 min period at 22-23°, triethylamine (107.5 g, 1.06 mol). The resulting mixture was stirred at 22-23° for 21 hr. During this time the mixture became thicker and a solution never formed. The mixture was concentrated by approximately 2.5 L and the residue was diluted with ethyl acetate (500 mL). The resulting thick suspension was stirred at −10° for 1 hr and filtered. The solid was rinsed with ethyl acetate (100 mL), pressed dry under suction and was further dried in vacuo at 36° for 7 hr. 162 g (71%) of product (8) was obtained.

¹H NMR (DMSO) ppm: 9.8 (s, 1H), 9.2 (s, 1H), 7.6-7.7 (m, 3H), 7.35-7.5 (m, 2H), 7.3 (m, 2H), 7.2 (d, 1H), 7.15 (s, 1H), 6.45 (d, 1H), 6.25 (t, 1H), 3.2 (2d, 2H), 1.6 (s, 3H).

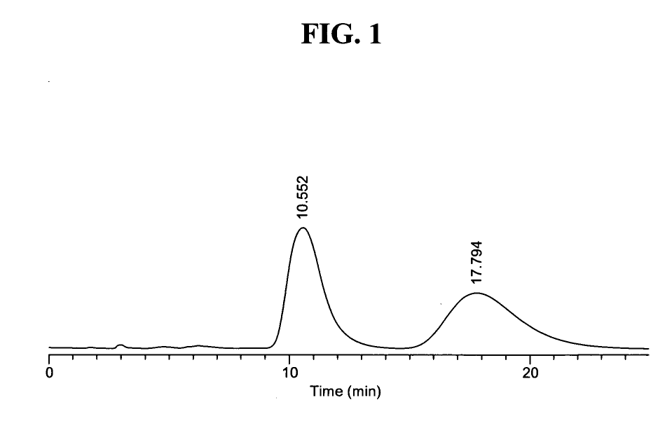

The product was analyzed on a Chiralpak AD (250×4.6 mm) at ambient temperature, the detector wavelength was 230 nm, the flow rate was 1.00 ml/min and the mobile phase was methanol. FIG. 1 shows the elution pattern of the two enantiomers as a function of time. The retention time for the (S)-enantiomer was 10.5 minutes and the retention time for the (R)-enantiomer was 17.8 minutes.

Step 5. Separation of (R) and (S) 5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide}.

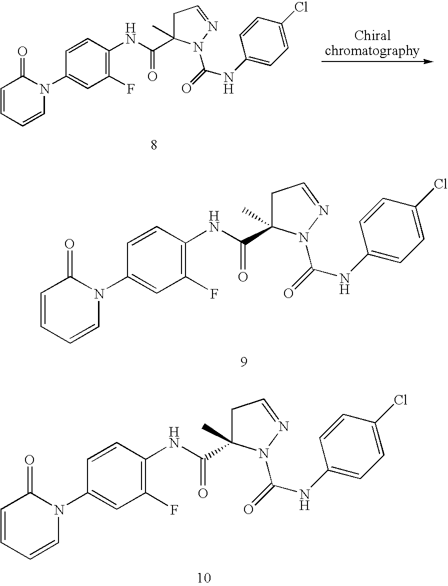

Procedure: A Biotage column was packed with ChiralPak AS resin. The mobile phase consisted of 80% methanol and 20% acetonitrile (v/v). The racemic feed consisted of 2.0 g of (8)/L in 75% acetonitrile and 25% methanol (v/v) with a flow rate of 1.5 L/min and a load of approximately 5.0 g/injection. The desired stream-second eluting compound was collected. From 492 g of racemate, 170 g of (9) was obtained following treatments with methylene chloride and ethylacetate.

The product was analyzed on a Chiralpak AD (250×4.6 mm) at ambient temperature, the detector wavelength was 230 nm, the flow rate was 1.00 ml/min and the mobile phase was methanol. Analysis showed that the material had 99.8% chiral purity.

¹H NMR (DMSO) ppm: 9.8 (s, 1H), 9.2 (s, 1H), 7.6-7.7 (m, 3H), 7.35-7.5 (m, 2H), 7.3 (m, 2H), 7.2 (d, 1H), 7.15 (s, 1H), 6.45 (d, 1H), 6.25 (t, 1H), 3.2 (2d, 2H), 1.6 (s, 3H).

### Example 2

(R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide}

Step 1. Synthesis of (3aS,6R,7aR)-1-methacryloyl-8,8-dimethylhexahydro-3a,6-methano-2,1-benzisothiazole 2,2-dioxide

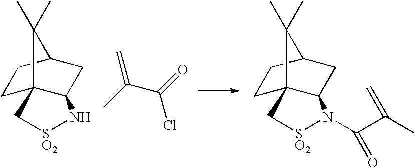

(1S)-(−)-2,10-camphorsultam

Procedure: To a solution of (1S)-(−)-2,10-camphorsultam (1.160 g, 5.388 mmoles) in toluene (10 ml) was added sodium hydride (60% in oil, 0.323 g, 8.08 mmoles). Stirred for 1.5 hours Added methacryoyl chloride (1.126 g, 10.78 mmoles) directly to the reaction mixture. Stirred at room temperature overnight, Evaporated, extracted into ethylacetate, washed with 1N HCl, dried MgSO₄, evaporated in vacuo and purified by chromatography (0-20% EtOAc in hexane) to afford the desired compound (1.240 g, 81%) 1H NMR (400 MHz, DMSO-D6) δ ppm 0.90 (s, 3H), 1.08 (s, 3H), 1.23 (m 1H), 1.42 (m, 1H), 1.78 (m, 5 H), 1.81 (m, 3H), 3.26 (s, 4H), 3.56 (d, J=14.04 Hz, 1H), 3.77 (d, J=14.04 Hz, 1H), 3.91 (m, 1H), 5.48 (s, 1H), 5.60 (s, 1H)

Step 2. Synthesis of (3aS,6R,7aR)-8,8-dimethyl-1-{[(5R)-5-methyl-4,5-dihydro-1H-pyrazol-5-yl]carbonyl}hexahydro-3a,6-methano-2,1-benzisothiazole 2,2-dioxide

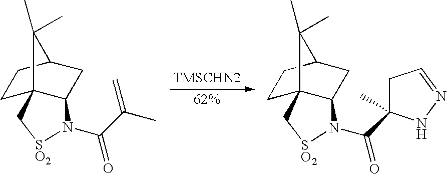

Procedure: To a solution of (trimethylsilyl)diazomethane in ether (2M; 20 ml) was added the solid (3aS,6R,7aR)-1-methacryloyl-8,8-dimethylhexahydro-3a,6-methano-2,1-benzisothiazole 2,2-dioxide and then the reaction mixture was stirred at room temperature for 72 hours. A solution formed. The solution was evaporated in vacuo and then diluted with methylene chloride (50 ml), cooled to 0° C. and then trifluoroacetic acid (1.2 ml) was added drop wise. The mixture was stirred for 2 h at 0° C. The reaction mixture was diluted with ethyl acetate (200 ml) and washed with sat. NaHCO₃ (200 mL). The organic phase was washed with brine (200 mL) and then dried with magnesium sulfate. The organic solvents were removed in vacuo and the product purified by silica gel chromatography (eluant 10-100 EtOAc in hexane) and then treated with ether. The product was isolated as a solid (2.90 g, 56% yield). Optical rotation=0.020 g in 2 ml; c=0.01 g/ml {C=1 (CHCl₃)}; measurement −0.287; optical rotation=−0.287×4000/10=−114.8 (using a Perkin-Elmer 241 Polarimeter).

### Combustion Analysis:

| | Carbon | Hydrogen | Nitrogen |
|---|---|---|---|
| Theory | 55.36 | 7.12 | 12.91 |
| Found | 55.29 | 7.09 | 12.83 |

1H NMR (400 MHz, DMSO-D6) δ ppm 0.87 (m, 3H), 0.98 (s, 3H), 1.21 (m, 1H), 1.32 (s, 3H), 1.40 (m, 1 H), 1.71 (m, 4 H), 1.85 (dd, J=13.35, 7.70 Hz, 1 H), 2.54 (dd, J=17.16, 1.56 Hz, 1 H), 3.13 (dt, J=17.20, 1.34 Hz, 1 H), 3.64 (d, J=−14.04 Hz, 1 H), 3.74 (d, J=14.04 Hz, 1 H), 3.83 (dd, J=7.60, 4.68 Hz, 1 H), 6.62 (t, J=1.46 Hz, 1 H), 6.78 (s, 1 H)

Step 3. Synthesis of (5R)-N-(4-chlorophenyl)-5-{[(3aS,6R,7aR)-8,8-dimethyl-2,2-dioxidotetrahydro-3a,6-methano-2,1-benzisothiazol-1(4H)-yl]carbonyl}-5-methyl-4,5-dihydro-1H-pyrazole-1-carboxamide

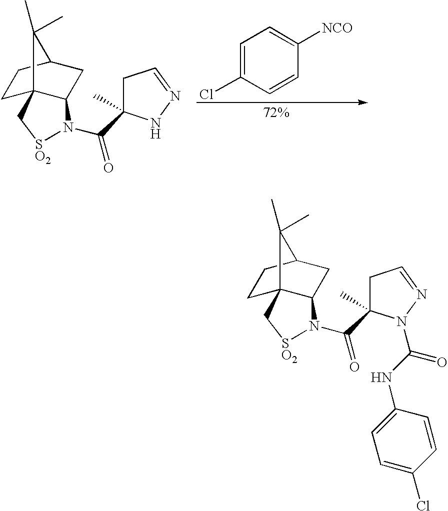

Procedure: To a solution of (3aS,6R,7aR)-8,8-dimethyl-1-{[(5R)-5-methyl-4,5-dihydro-1H-pyrazol-5-yl]carbonyl}hexahydro-3a,6-methano-2,1-benzisothiazole 2,2-dioxide (0.510 g, 1.56 mmoles) in methylene chloride (2 ml) was added sat. sodium bicarbonate (1 ml) and then 4-chlorophenyl isocyanate (0.530 g, 3.44 mmoles). The mixture was stirred at room temperature for 3 hours. The mixture was diluted with methylene chloride (20 mL) and then filtered and concentrated. Purification by silica gel chromatography (eluant 5-100% EtOAc in hexane) affords the required compound (0.740 g). This solid was crystallized from CH₂Cl₂/methyl t-butylether. The solid was combined with a second crop from the mother liquors to afford the title compound (0.540 g, 72% yield)

Optical rotation=0.020 g in 2 ml; c=0.01 g/ml {C=1 (CHCl₃)}; measurement −0.120; optical rotation=−0.120×4000/10=−48.0

### Combustion Analysis:

| | Carbon | Hydrogen | Nitrogen |
|---|---|---|---|
| Theory | 55.17 | 5.68 | 11.70 |
| Found | 55.22 | 5.54 | 11.60 |

1H NMR (400 MHz, DMSO-D6) δ ppm 0.86 (s, 3 H), 1.01 (s, 3 H), 1.21 (m, 1 H), 1.41 (m, 1H), 1.46 (s, 3 H) 1.72 (m, 3 H) 1.85 (m, 2 H), 2.86 (dd, J=17.93, 1.75 Hz, 1 H) 3.24 (dd, J=17.93, 1.75 Hz, 1 H), 3.61 (d, J=14.04 Hz, 1 H), 3.70 (d, J=14.04 Hz, 1 H), 3.87 (dd, J=7.02, 5.46 Hz, 1 H), 5.15 (s, 1 H), 6.49 (m, 1 H), 7.22 (d, J=9.0 Hz, 2 H), 7.57 (d, J=9.0 Hz 2 H), 9.07 (s, 1 H)

Step 4: Synthesis of (5R)-1-{[(4-chlorophenyl)amino]carbonyl)-5-methyl-4,5-dihydro-1H-pyrazole-5-carboxylic acid

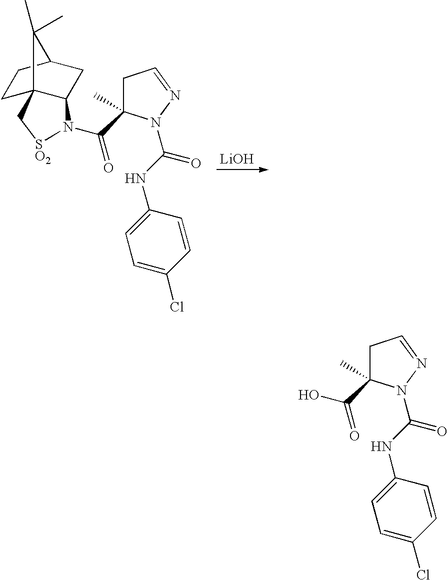

Procedure: Dissolved (5R)-N-(4-chlorophenyl)-5-{[(3aS,6R,7aR)-8,8-dimethyl-2,2-dioxidotetrahydro-3a,6-methano-2,1-benzisothiazol-1(4H)-yl]carbonyl}-5-methyl-4,5-dihydro-1H-pyrazole-1-carboxamide (0.500 g, 1.04 mmoles) in MeOH (1 ml), THF (1 ml), H₂O (2 ml). Added solid LiOH.H₂O (0.094 g, 2.24 mmoles) and stirred for 3 hours at room temperature. Diluted with water (20 ml) and then added 1 N HCl to adjust the pH to 5. The reaction mixture was filtered to remove the sultam. Aqueous phase was lyophilsed to afford the desired product as the lithium salt contaminated with approx. 20% of the sultam.

1H NMR (400 MHz, DMSO-D6) δ ppm 1.53 (s, 3 H), 2.92 (dd, J=18.62, 1.66 Hz, 1 H), 3.19 (dd, J=18.62, 1.66 Hz, 1 H), 7.02 (t, J=1.66 Hz, 1 H) 7.25 (m, 2 H) 7.62 (m, 2 H) 9.10 (s, 1 H)

Step 5: Synthesis of (R)-5-Methyl-4,5-dihydro-pyrazole-1,5-dicarboxylic acid 1-[(4-chloro-phenyl)amide] 5-{[2-fluoro-2-oxo-2H-pyridin-1-yl)-phenyl]-amide}

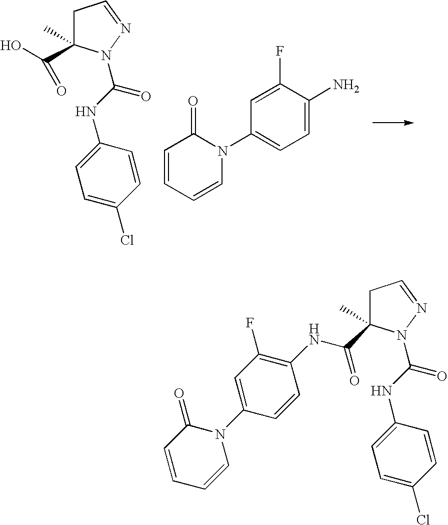

Procedure: A 1.0M solution of thionyl chloride in dry methylene chloride (0.319 mL, 0.319 mmol) was added to a solution of DMAP (43.5 mg, 0.213 mmol) in dry THF (2 mL) previously cooled to −20° C. (ice-NaCl-water-acetone-dry ice). The mixture was stirred for 5 min and then a solution of (5R)-1-{[(4-chlorophenyl)amino]carbonyl}-5-methyl-4,5-dihydro-1H-pyrazole-5-carboxylic acid (0.030 g, 0.11 mmol) in THF (1 mL) was added. The mixture was stirred for 20 min and a solution of aniline (0.049 g, 0.24 mmol) in THF (4 mL) was added. The mixture was allowed to warm slowly to 23° C. over 2 hours and then stir at 23° C. for 16 hours. This reaction mixture was combined with a duplicate run of the reaction. Water (5 mL) was added and the THF evaporated. The residue was partitioned between DCM (80 mL) and 10% HCl (20 mL). The phases were separated. The aqueous phase was extracted with DCM (2×20 mL). The combined organic extracts were washed with 5% HCl (1×20 mL), 0.5 N NaOH (1×20 mL), and brine (1×20 mL), dried over MgSO4, filtered and solvent removed. The residue was purified by MPLC under the following conditions: Column: RediSep 40 g; Eluant: 0% EtOAc in Heptane to 100% EtOAc in Heptane in 15 min, then 0% MeOH in EtOAc to 5% MeOH in EtOAc in 45 min. The pure fractions, as determined by TLC, were combined and solvent removed. The residues were dried under high vacuum overnight. This afforded 0.079 g of a white solid (74% yield for combined reactions). HPLC purity for this compound was 98.46%. Further purification was done by trituration with diethyl ether for 7 h. Upon filtration a white solid was collected (0.059 g).

### Combustion Analysis:

| | Carbon | Hydrogen | Nitrogen |
|---|---|---|---|
| Theory | 59.04 | 4.09 | 14.97 |
| Found | 58.64 | 3.91 | 14.61 |

1H NMR (400 MHz, CHLOROFORM-d) δ ppm 1.88 (s, 3 H) 2.89 (dd, J=19.20, 1.66 Hz, 1 H) 4.10 (dd, J=19.30, 1.75 Hz, 1 H) 6.21 (td, J=6.73, 1.17 Hz, 1 H) 6.61 (d, J=8.77 Hz, 1 H) 6.92 (t, J=1.56 Hz, 1 H) 7.11 (dd, J=8.77, 1.36 Hz, 1 H) 7.20-7.29 (m, 4 H) 7.31-7.39 (m, 1 H) 7.41-7.46 (m, 2 H), 8.14 (s, 1H), 8.39 (t,, J=19.2 Hz, 1 H), 10.83 (s, 1 H)

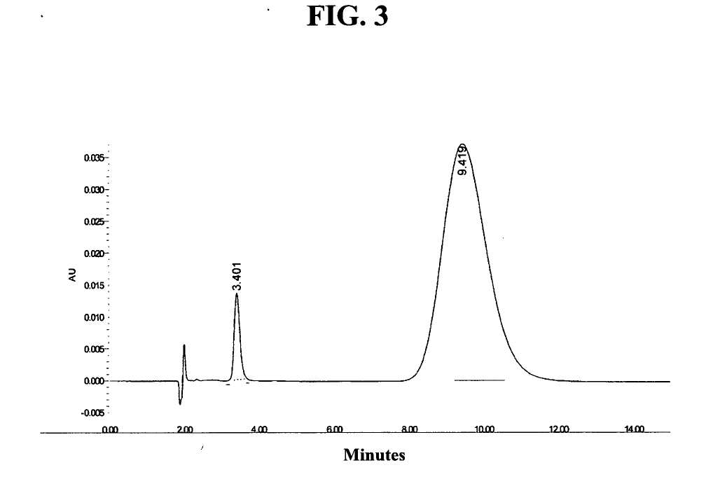

The final product was run over a Chiralcel OJ-R (150×4.6 mm) 10 um, at ambient temperature, detector wavelength 252 nm, flow rate 1.00 ml/min, mobile phase methanol The material assayed at 95% (9) and 5% (10). FIG. 3 shows the elution pattern as a function of time. The retention time for the (S)-enantiomer was 3.4 minutes and the retention time for the (R)-enantiomer was 9.4 minutes.

To improve the optical purity of the final compound a chiral HPLC preparation may be performed for example, with a Chiralpak AS column.

### Example 3

Step 1. Synthesis of (3aS,6R,7aR)-1-methacryloyl-8,8-dimethylhexahydro-3a,6-methano-2,1-benzisothiazole 2,2-dioxide.

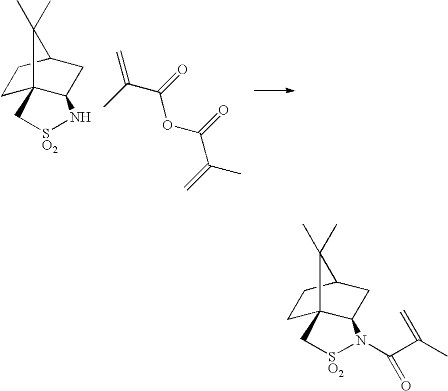

(1S)-(−)-2,10-camphorsultam

Procedure: To a solution of the (1S)-(−)-2,10-camphorsultam (5.000 g, 23.22 mmoles) in anhydrous THF (50 ml) at −20° C. was added lithium chloride (1.08 g, 25.5 mmoles,—small balls), triethylamine (4.21 ml, 30.2 mmoles, 1.30 equivalents.) and then the mixture was allowed to stir for 10 minutes. The lithium chloride did not go into solution. 2-methylacrylic anhydride (4.15 ml, 27.9 mmoles, 1.20 equivalents.) in THF (15 ml) was then added. The internal temperature varied between −20° C. and −10° C. during addition which took about 5 minutes. The thick white mixture was stirred within the cooling bath and allowed to reach room temperature. After stirring overnight the white heterogeneous reaction mixture was added to 350 ml of water with stirring. The white crystalline solid (3aS,6R,7aR)-1-methacryloyl-8,8-dimethylhexahydro-3a,6-methano-2,1-benzisothiazole 2,2-dioxide (6.302 g, 96% yield) was collected and dried under vacuum.

Analytical HPLC run with a Vydac 218TP54 C18 reverse phase column run with solvents A: 0.1% trifluoroacetic acid in H2O and B: 0.1% trifluoroacetic acid in acetonitrile. Gradient (0 to 100%B) over 22 minutes. Retention time 18.166 min (100%).

Optical rotation=0.020 g in 2 ml; c=0.01 g/ml {C=1 (CHCl3)}; measurement −0.226; optical rotation=−0.226×4000/10=−90.4.

### Combustion Analysis:

| | Carbon | Hydrogen | Nitrogen |
|---|---|---|---|
| Theory | 59.34 | 7.47 | 4.94 |
| Found | 59.46 | 7.52 | 4.82 |

Steps 2 through 5 were performed as in Example 2.

### Example 4

IC₅₀ of the (R) enantiomer

The assay was performed in triplicate using a final concentration of 30 pM of human factor Xa (diluted in buffer containing 10 mM HEPES, 150 mM NaCl, 0.1% bovine serum albumin (BSA), pH 7.4). The substrate, S-2765 (N-CBz-D-Arg-L-Gly-L-Arg-p-nitroanilide, 2HCl), was run at a final concentration equal to its Kₘ value. The (R) enantiomer was serially diluted in 100% DMSO (final assay concentration=2% DMSO) to final concentrations of 1.0 μM to 958 μM. Dilutions were transferred to a black polystyrene 96-plate microtiter assay plate at a volume of 2.5 μL/well. Buffer containing the human FXa was added to each well at a volume of 73 μL/well and then gently shaken while incubating at room temperature for 50 minutes. At the end of 50 minutes, both the plate and buffer-diluted substrate were incubated at 37° C. for 10 minutes. The reactions were initiated by the addition of 50 μL of pre-warmed substrate and immediately placed in the microplate reader pre-warmed at 37° C.

A fluorometric plate reader was used to continuously monitor the rate of change in relative fluorescence units (RFUS) per unit time. The assay plate was mixed once before reading. The fluorogenic emission was read every 44 seconds for 30 minutes (excitation/emission of λₑₓ=390 nm and λₑₓ=460 nm; automatic cutoff=455 nm) at 37° C.

What is claimed is:

1. The compound (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide}, wherein the (R) enantiomer is substantially pure.

2. The compound according to claim 1 wherein the ratio of the (R) enantiomer to the (S) enantiomer is greater than 4:1

3. The compound according to claim 1 wherein the ratio of the (R) enantiomer to the (S) enantiomer is greater than 19:1.

4. The compound according to claim 1 wherein the ratio of the (R) enantiomer to the (S) enantiomer is greater than 99:1.

5. A pharmaceutical composition comprising a pharmaceutically acceptable carrier, excipient or diluent and (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide} wherein the (R) enantiomer is substantially pure.

6. The pharmaceutical composition of claim 5 wherein the ratio of the (R) enantiomer to the (S) enantiomer is greater than 4:1.

7. The pharmaceutical composition according to claim 5 wherein the ratio of the (R) enantiomer to the (S) enantiomer is greater than 19:1.

8. The pharmaceutical composition according to claim 5 wherein the ratio of the (R) enantiomer to the (S) enantiomer is greater than 99:1.

9. A method for the treatment of a thrombotic, or embolic disorder in a patient in need thereof, comprising administering a therapeutically effective amount of (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide} wherein the (R) enantomer is substantially pure.

10. The method of claim 9 wherein the ratio of the (R) enantiomer to the (S) enantiomer is greater than 4:1.

11. The method of claim 9 wherein the ratio of the (R) enantiomer to the (S) enantiomer is greater than 19:1.

12. The method of claim 9 wherein the ratio of the (R) enantiomer to the (S) enantiomer is greater than 99:1.

13. The method of claim 9, wherein the thrombotic or embolic disorder is primary or secondary venous thrombosis, arterial thrombosis, venous embolism, arterial embolism, venous stenosis, venous restenosis, arterial stenosis or arterial restenosis.

14. The method of claim 9 wherein treatment is of a patient with atrial fibrillation, angina, diabetes, cancer or heart failure, or immobilization due to trauma, surgery or medical illness.

15. The method of claim 9 wherein the patient is a patient with primary DVT, secondary VTE or atrial fibrillation.

16. The method of claim 9 wherein the treatment is to prevent pulmonary embolism, pulmonary hypertension, or stroke.

17. A method for the treatment of a patient with a metastatic disorder in order to prolong survival in a patient in need thereof comprising administering a therapeutically effective amount of comprising (R)-5-Methyl-4,5-dihydro-pyrazole-1,5-dicarboxylic acid 1-[(4-chloro-phenyl)amide] 5-{[2-fluoro-4-(2-oxo-2H-pyridin-1-yl)-phenyl]-amide} wherein the ratio of the (R) enantiomer to the (S) enantiomer is greater than 4:1.

18. A method for the treatment of sepsis in a patient in need thereof, comprising administering a therapeutically effective amount of comprising (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide} wherein the ratio of the (R) enantiomer to the (S) enantiomer is greater than 4:1.

19. The use of substantially pure (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl]-amide for the manufacture of a medicament for the therapeutic treatment or prophylactic treatment of thrombotic disorders in mammals.

20. A method for making (R)-5-Methyl-4,5-dihydro-pyrazole-1,5-dicarboxylic acid 1-[(4-chloro-phenyl)-amide] 5-{[2-fluoro-4-(2-oxo-2H-pyridin-1-yl)-phenyl] 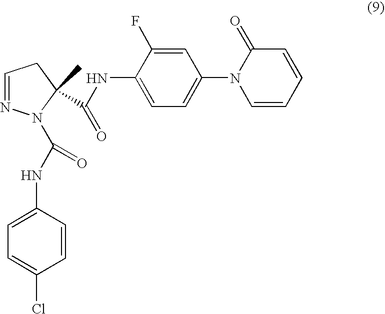
comprising:
Step (a)
(1) reacting the compound of Formula 11 with methacryoyl chloride in the presence of a solvent and a base; or
(2) reacting a compound of Formula 11 with lithium chloride, triethylamine and 2-methylacrylic anhydride; 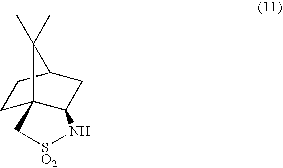
to give a compound of Formula 12 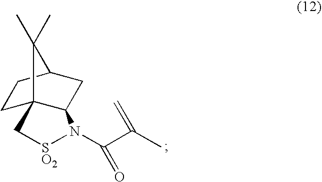
Step (b) reacting the compound of Formula 12 with trimethylsilyl diazomethane, treated with dilute acid and isolated to afford a compound of Formula 13 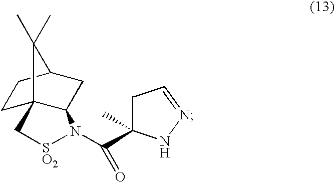
Step (c) treating the compound of Formula 13 with an isocyanate and a mild base in a solvent to give a compound of Formula 14 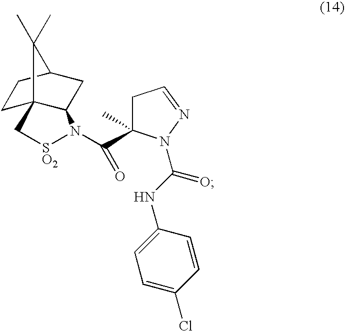
Step (d) removing the chiral auxiliary to give a compound of Formula 15 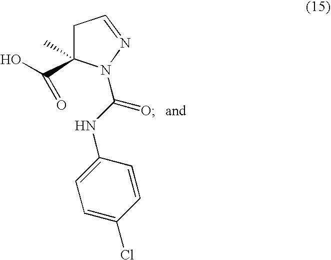
Step (e) coupling the compound of Formula 15 with a compound of Formula 3 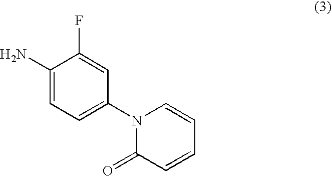
to afford the desired compound.

21. A crystalline form having a powder X-ray diffraction pattern with at least one peak at 5.4, 7.2, 9.4, 10.9, 15.6, 19.6. 21.7 or 23.3 degrees 2θ;

22. A crystalline form having a powder X-ray diffraction pattern with peaks at 19.6 and 21.7 and one or more additional peaks at 7.2, 9.4, 10.9, 15.6 or 23.3 degrees 2θ.

23. A crystalline form having a powder X-ray diffraction pattern with peaks at peaks at 10.9, 19.6 and 21.7 and one or more additional peaks at 7.2, 9.4, 15.6 or 23.3 degrees 2θ.
